(19) United States
(12) Patent Application Publication
(10) Pub. No.: US 2026/0062705 A1
(43) Pub. Date: Mar. 5, 2026

(54) TREATMENT FOR OVARIAN CANCER WITH OLIGONUCLEOTIDES TARGETING FIBROBLAST ACTIVATION PROTEIN ALPHA

(71) Applicant: UNIVERSITY OF FLORIDA RESEARCH FOUNDATION, INCORPORATED, Gainesville, FL (US)

(72) Inventors: Shuang HUANG, Gainesville, FL (US); Bin LI, Gainesville, FL (US)

(21) Appl. No.: 19/106,799

(22) PCT Filed: Aug. 31, 2023
(86) PCT No.: PCT/US2023/031635

§ 371 Date: Feb. 26, 2025

Related U.S. Application Data
(60) Provisional application No. 63/374,045, filed on Aug. 31, 2022.

(51) Int. Cl. C12N 15/113; A61K 45/06; A61P 35/00; C12N 15/115

(52) U.S. Cl. CPC C12N 15/1137; A61K 45/06; A61P 35/00; C12N 15/115; C12N 2310/14; C12N 2310/16; C12N 2310/322; C12N 2310/3519; C12Y 304/14005

(57) ABSTRACT

Fibroblast activation protein a (FAP) is a tumor-specific protein and well characterized for its function in tumorigenicity. However, agents against its enzymatic activity or monoclonal antibodies against its cell surface presence are unsuccessful in clinic for uncharacterized reason. In this disclosure, provided is an anti-FAP siRNA and FAP-silencing oligonucleotides comprising anti-FAP siRNAs linked at both ends to aptamers recognizing cancer markers, in particular aptamers recognizing epithelial cell adhesion molecule (EpCAM).

### GOVERNMENT FUNDING SUPPORT

This invention was made with government support under grant No. CA222467 and CA256482 awarded by The National Institutes of Health. The government has certain rights in the invention.

### BACKGROUND

### Field of the Invention

The invention relates to the field of medicine, in particular to treatment for cancers, in particular to treatment for cancers sensitive to the knockdown of fibroblast activation protein alpha (FAP). Preferred embodiments of the invention relate to treatments for ovarian cancer.

### Background of the Invention

Ovarian cancer is the deadliest gynecological cancer, and its high mortality rate is mainly due to the lack of symptom at early stage. In fact, stage I and II patients have 5-year survival of 90% and over 70% respectively while 5-year survival rate drops to 39% for stage III patients and dismal 17% for stage IV patients. Current mainstay of ovarian cancer therapy is the combination of surgery and chemotherapy. However, majority of patients will develop therapeutic resistance, suffer relapse and eventually succumb to the disease. It is reasoned that finding ways to deter the late-stage events can improve clinical outcome of ovarian cancer patients, and this should be facilitated by identifying molecules/pathways key for ovarian cancer progression and survival.

### SUMMARY

In order to tackle such unmet need to treat ovarian cancer and find genes upregulated in advanced ovarian cancer, we performed the analysis of the Cancer Genome Atlas (TCGA) ovarian cancer data set, and discovered FAP is one of those upregulated proteins. Thus, in this invention we provide oligonucleotides comprising anti-FAP siRNAs, in particular anti-FAP siRNAs linked to aptamers that recognize and bind to a cancer marker protein, in particular to epithelial cell adhesion molecule (EpCAM).

FAP is a 97-kDa type II transmembrane serine proteinase and a member of the prolyl peptidase family, which also includes dipeptidyl peptidase IV (DPPIV, CD26), DPP7 (DPP II, quiescent cell proline dipeptidase), DPP8, DPP9, and prolyl carboxypeptidase (PCP, angiotensinase C). Like many other members of the family, FAP has dipeptidyl peptidase enzymatic activity, but FAP also has endopeptidase activity (gelatinase activity) (Fitzgerald and Weiner. 2020; Goldstein et al.; 1997; Rosenblum. 2003; Collins et al., 2004; Edosada et al, 2006; Aggarwal et al. 2008; Huang et al, 2011).

FAP is usually absent in most normal adult tissues, but FAP expression has been reported to be elevated under pathological conditions such as fibrosis, arthritis and cancer, especially in tumor stroma (cancer-associated fibroblasts). Actually, elevated FAP expression and activation were shown in various cancers such as adenocarcinoma of the colon and stomach, invasive ductal carcinoma of the breast, and malignant melanoma. In addition, high FAP expression seems negatively associated with the overall survival rate of the patients. However, the biological mechanisms underlying its clinical association with disease severity remain unclear, and it is suggested that FAP may affect clinical outcomes via its effects on extracellular matrix remodeling, intracellular signaling regulation, angiogenesis, epithelial-to-mesenchymal transition and immunosuppression (Fitzgerald and Weiner, 2020).

FAP has also been reported to be present in almost all ovarian cancer, but not in normal ovary tissue and benign ovarian tissues. FAP expression in ovary cancer stroma is certain, and its expression was also detected in ovarian cancer cell lines. Experimental evidence from both in vitro and in vivo studies showed that the proteinase activity of FAP may be a key contributor to its cancer-promoting role though there are other reports indicating that the proteinase activity of FAP may not play a critical role in cancer cell growth. In an embodiment of this invention, we also showed that a proteinase-inactivated FAP behaved similarly to wild-type FAP in ovarian cancer cell growth, proving that the enzymatic role of FAP is not essential for cancer promotion.

Because of such FAP's tumor-specific expression and tumor-promoting capability, efforts have been exerted to develop small molecular inhibitors targeting FAP activity and monoclonal antibodies targeting its cell surface presence. Unfortunately, clinic trials with these agents have not been unsuccessful and the underlying reasons have not been addressed. Therefore, there is a need for a new therapeutic approach to silence/suppress FAP gene and/or protein to cure cancers, in particular ovarian cancer, and anti-FAP siRNA can be one of such approaches.

However, when the matter concerns with the targeting strategies of a therapeutic agent such as siRNA, there is a need for target-specific delivery since insufficient delivery of it into tumor tissues results in inadequate distribution and low intracellular concentration of the therapeutic agent, leading to failure of cancer treatment. Thus, targeted delivery of therapeutic agent such as siRNA to tumors followed by complete internalization will significantly improve clinical outcomes of cancer therapy.

For cancer therapy using siRNA, various effective delivery systems have been designed for specific delivery of functional siRNA into the target cancer cells to achieve an effective intracellular concentration. A suitable targeting ligand is usually added or conjugated to the carrier to achieve tumor-specific siRNA delivery.

Among them, monoclonal antibodies conjugated to siRNA have been commonly used as ligand for cancer cell specific delivery, but the penetration of such siRNA into tumor tissues is limited because of the large size of the ligand monoclonal antibodies. In addition, it takes time, efforts and costs to create such a siRNA-monoclonal conjugation. In this aspect, aptamers, “chemical antibodies”, which are much smaller than antibodies and capable of selective binding to specific targets are being considered as an alternative therapeutic strategy. In addition, chemically synthesized aptamers can be reproducibly scaled-up, and they are more readily modified with functional groups to meet specific therapeutic and diagnostic needs. Thus, aptamers are more effective therapeutic agents than monoclonal antibodies.

In Xiang et al. published in 2015, it was demonstrated that aptamers were superior to antibodies in terms of tumor penetration, by comparing the performance of an EpCAM aptamer with an EpCAM antibody, especially in penetration and retention in vivo live animal imaging and mouse colorectal cancer xenograft model. Thus, aptamers seem promising molecules for targeted tumor therapeutics.

Therefore, in some embodiments for effective delivery of anti-FAP siRNA in vivo in order to knockdown or silence FAP gene in ovarian cancer, an anti-FAP siRNA is linked to RNA aptamers targeting a cancer marker protein on the cancer cell surface, in particular to EpCAM aptamers. EpCAM is highly expressed in ovarian cancer but not in other tissues in peritoneal cavity, and it was believed that EpCAM aptamer might be a suitable vehicle for FAP siRNA delivery.

The epithelial cell adhesion molecule (EpCAM, CD326) is a glycoprotein of ˜40 kd, and non-cancerous normal epithelial cells generally express EpCAM at a lower level than cancer cells (Trzpis et al., 2007). EpCAM was originally identified as a marker for carcinoma due to its high expression on rapidly proliferating cancers, especially on tumors of epithelial origin. In early studies, the role of EpCAM was suggested to be a cell-cell adhesion molecule. However, many other recent studies proposed the roles for EpCAM not only in cell adhesion but also in diverse processes such as cell signaling, migration, proliferation, and differentiation (Litvinov et al., 1997; Cirulli et al, 1998; Nochi et al., 2004; Osta et al., 2004). Further, there have been the implications of the newly identified functions of EpCAM in view of its relevance in carcinoma of epithelial origin. However, in some embodiments of this invention, it is demonstrated that EpCAM aptamer can also be used for mesenchymal-origin ovarian cancer cells, and demonstrated that an anti-FAP siRNA linked to EpCAM aptamers can effectively suppress mesenchymal-like ovarian cancer development.

Thus, in certain embodiments, this aspect of an aptamer is exploited by providing oligonucleotides comprising anti-FAP siRNAs linked to EpCAM aptamers, in particular anti-FAP siRNA linked to EpCAM aptamers at both ends of the dsRNA siRNA, in which one end (5′- or 3′ end-) of an anti-FAP siRNA sense strand is linked to an EpCAM aptamer and the other end (5′- or 3′ end-) of anti-FAP siRNA antisense strand is linked to another EpCAM aptamer to form an aptamer-anti-FAP (ds) siRNA-aptamer chimera. In some embodiments, an oligonucleotide comprising an anti-FAP siRNA and two EpCAM aptamers is selectively taken up and reduced FAP gene expression in cancer cells in vitro and ovarian tumor size in vivo (FIG. 7).

In summary, to cure ovarian cancer, in this invention we provide oligonucleotides comprising an anti-FAP-siRNA to knock down or silence FAP gene, in particular an anti-FAP-siRNA linked to cancer marker-recognizing/binding aptamers, in particular to EpCAM aptamers.

### DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1A is a volcano plot to identify genes differentially expressed in advanced stage of ovarian cancer (Stage III/IV vs I/II).

FIG. 1B is a graph showing the level of FAP mRNA in normal ovary and ovarian cancer. (N, number of patients. ****, P<0.0001)

FIG. 1C is a graph showing the level of FAP mRNA in Stage I, II, III and IV ovarian cancer patients. (N, number of patients. **, P<0.01)

FIG. 1D is a graph showing Kaplan-Meier overall survival plots of FAP.

FIG. 1E is a graph showing Kaplan-Meier progression-free survival plot.

FIG. 1F is a western blot showing the levels of FAP, vimentin (mesenchymal marker), E-cadherin (epithelial marker) and GAPDH (control) after overnight cell culture.

FIG. 1G is a graph showing invasiveness of the cells cultured in Matrigel invasion chamber. Overnight-cultured cells were plated into Matrigel invasion chambers and allowed to invade for 24 h. Cells on undersurface of invasive chambers were stained while cells remaining in the chambers were removed. Stained cells were solubilized and measured using plate reader. Data are means±SD.

FIG. 2A is a graph showing CCK-8 viability assay of the cells lentivirally transduced with cither scrambled sequence (control) or FAP shRNA for 4 days. Data are means±SD. ****, P<0.0001; n.s., no significance.

FIG. 2B is a graph showing CCK-8 viability assay. HEY and OVCAR8 cells were first lentivirally transduced with FAP or FAP/S624 (a mutant without proteinase activity) for 1 day and further transduced with control or FAP shRNA for additional 4 days. Data are mean±SD. ****, P<0.0001.

FIG. 2C presents Annexin V/PI-based flow cytometry of HEY and OVCAR8 cells lentivirally transduced with scramble or FAP shRNA for 4 days. The horizontal axis is Annexin V staining and the vertical axis is PI staining. Cells in Quadrants 2 and 3 represent apoptotic cells. Results are representative of three independent experiments.

FIG. 2D is a western blot showing the levels of cleaved PARP, cleaved caspase-3 and GAPDH of HEY and OVCAR8 cells infected with lentiviral vector containing scramble or FAP shRNA for 4 days.

FIG. 2E is a graph showing CCK-8 viability assay. HEY and OVCAR8 cells were transduced with lentivirus containing scramble or FAP shRNA. One day post-infection, cells transduced with FAP shRNA were cither untreated or treated with vehicle (DMSO), 10 μM zVAD or 10 μM DEVD. After total of 4 days, cell viability was analyzed by CCK-8 assay. Data are means±SD. ****, P<0.0001.

FIG. 3A and FIG. 3B are the results of total RNA sequencing of OVCAR8 cells lentivirally transduced with scramble (control) or FAP shRNA. Data were analyzed by web-based Gene Sequence Enrichment Analysis (A) and Ingenuity Pathway Analysis (B).

FIG. 3C is a graph showing luciferase activity measurement. HEY and OVCAR8 cells were lentivirally transduced with scramble or FAP shRNA for 2 days and then transfected with NF-κB promoter reporter construct for another day. Data are mean±SD. ****, P<0.0001.

FIG. 3D is a graph showing ELISA to measure the amount of individual NF-κB family member in nuclei of OVCAR8 cells lentivirally transduced with scramble or FAP shRNA. Data are means±SD. ****, P<0.0001.

FIG. 3E is a graph showing the levels of FAP and REL. HEY and OVCAR8 cells were lentivirally transduced with scramble or FAP shRNA for 2 days followed by total RNA extraction. RNA was subjected RT-qPCR to measure the levels of FAP and REL using specific primer sets. β-Actin mRNA was used as an internal control for standardization. Data are means±SD. ****, P<0.0001.

FIG. 3F is a western blot showing FAP, REL and GAPDH in HEY and OVCAR8 cells transduced with scramble or FAP shRNA.

FIG. 3G is a graph showing luciferase activity measurement. HEY and OVCAR8 cells were lentivirally transduced with scramble or REL shRNA for 2 days and then transfected with NF-κB promoter reporter construct for another day. Data are mean±SD. ****, P<0.0001.

FIG. 3H is a graph showing CCK-8 viability assay. HEY and OVCAR8 cells were lentivirally transduced with scramble, REL, RELA or RELB shRNA for 4 days. Data are mean±SD. ****, P<0.0001.

FIG. 4A is a western blot showing FAP and pro-survival proteins. HEY and OVCAR8 cells were lentivirally transduced with scramble or FAP shRNA for 3 days and then lysed for western blot analysis with the respective antibodies.

FIG. 4B is a western blot showing REL, BIRC5 and GAPDH. HEY and OVCAR8 cells were lentivirally transduced with scramble or REL shRNA for 3 days and then lysed for western blot analysis with the respective antibodies.

FIG. 4C is a graph showing CCK-8 viability assay of HEY and OVCAR8 cells lentivirally transduced with scramble or BIRC5 shRNA for 4 days. Data are mean±SD. ****, P<0.0001.

FIG. 4D is a graph showing CCK-8 viability assay of HEY and OVCAR8 cells treated with vehicle, 100 nM YM155, 100 nM Birinapant or 100 nM S63845 for 4 days. Data are mean±SD. ****, P<0.0001.

FIG. 5A is a graph showing CCK-8 viability assay of HEY and OVCAR8 cells lentivirally transduced with scramble or PRKDC shRNA for 4 days. Data are mean±SD. ****, P<0.0001.

FIG. 5B is a graph showing luciferase activity measurement. HEY and OVCAR8 cells were lentivirally transduced with scramble or PRKDC shRNA for 2 days and then transfected with NF-κB promoter reporter construct for another day. Data are mean±SD. ****, P<0.0001.

FIG. 5C is a western blot showing PRKDC, FAP, REL, BIRC5 and GAPDH. HEY and OVCAR8 cells were lentivirally transduced with scramble or PRKDC shRNA for 3 days and then lysed for western blot analysis with the respective antibodies.

FIG. 5D is a western blot showing phosphor-AKT (Ser473), AKT and GAPDH in HEY and OVCAR8 cells transduced with scramble or PRKDC shRNA.

FIG. 5E is a western blot showing phosphor-AKT (Ser473), AKT and GAPDH in HEY and OVCAR8 cells transduced with scramble or FAP shRNA.

FIG. 5F is a graph showing CCK-8 viability assay of HEY and OVCAR8 cells treated with vehicle (DMSO) or 1 μM AZD5363 for 2 days. Data are means±SD. ****, P<0.0001.

FIG. 6A is the results of immunoprecipitation with FAP and PRKDC antibodies. HEY and OVCAR8 cells were immunoprecipitated with either PRKDC or FAP antibody and the immunoprecipitates were subjected to western blot to detect FAP or PRKDC respectively. All blots derived from the same experiment and were processed in parallel.

FIG. 6B is immunofluorescence staining to detect FAP and PRKDC in HEY and OVCAR8. Data are the representative of six independent experiments.

FIG. 6C is a western blot showing PRKDC, FAP and caveolin-1. Lipid rafts were isolated from HEY and OVCAR8 cells and subjected to western blot analysis with the respective antibodies.

FIG. 6D is lipid raft staining showing FAP and PRKDC. HEY and OVCAR8 cells were fixed and then stained for FAP and PRKDC with respective antibodies. Labeled Cholera toxin subunit was also included for lipid raft staining. Data are the representative of six independent experiments.

FIG. 6E is immunofluorescence staining showing FAP and PRKDC in HEY and OVCAR8 cells treated with 5 mM MBC for 1 day. Data are the representative of six independent experiments.

FIG. 6F is a western blot showing PRKDC and caveolin-1. HEY and OVCAR8 cells were lentivirally transduced with scramble or FAP shRNA for 3 days and then subjected to lipid raft isolation. Western blot analysis was performed with these isolates.

FIG. 6G is immunofluorescence staining showing PRKDC and lipid rafts in HEY and OVCAR8 cells lentivirally transduced with scramble or FAP shRNA for 3 days. Data are the representative of six independent experiments.

FIG. 7A is an exemplary structure of EpCAM aptamer-FAP siRNA chimera.

FIG. 7B is a graph showing CCK-8 viability assay of HEY and OVCAR8 cells treated with scramble control or EpCAM-Apt/FAP siRNA. Data are means±SD. ****, P<0.0001.

FIG. 7C and FIG. 7D show luciferase-expressing cells. Luciferase-expressing HEY (C) or OVCAR8 cells (D) (106 cells/mouse) were intraperitoneally inject into female athymic nude mice. One week after tumor cell injection, mice were divided into two groups: one was treated with scramble aptamer and the other with EpCAM-Apt/FAP siRNA. Intraperitoneal xenograft development was monitored weekly using the Xenogen IVIS-200 In Vivo bioluminescence imaging system. Left panel: xenograft development curve. Data are means±SD. n=5. *, P<0.05; **, P<0.01; ***, P<0.001; ****, P<0.0001. Right panel: Bioluminescent images of the xenograft tumors at the beginning (week 1) and end of treatment (week 5). Two representative mice from each group are shown. The image data is displayed in radiance or photons/sec/cm2/steradian.

FIG. 7E is images of sacrificed mice after 4 weeks of treatment. Tumor are circled in white.

FIG. 7F is a graph showing tumor weight of the implanted tumors. Data are means±SD. **, P<0.01.

FIG. 7G is representative pictures of IHC staining of FAP, TUNEL and cleaved caspase-3 in tumor tissues derived from HEY and OVCAR8 cells.

FIG. 8 Effectiveness of designed FAP shRNAs on FAP level. HEY cells were lentivirally transduced with scramble or various designed FAP shRNAs for 2 days and then subjected to RT-qPCR to measure FAP mRNA (FIG. 8A) and to western blotting to detect FAP protein (FIG. 8B).

FIG. 9 Effect of FAP enzymatic inhibitors on ovarian cancer cell viability. HEY and OVCAR8 cells were treated with 1 mM Batimastat or 1 μM Talabostat alone or in combination for 4 days and then subjected to CCK-8 assay to assess cell viability, Data are means±SD.

FIG. 10 Effect of FAP knockdown on NF-κB activity in ovarian cancer cells. FIG. 10A. ELISA to measure the amount of individual NF-κB family member in nuclei of HEY cells lentivirally transduced with scramble or FAP shRNA. Data are means±SD. ****, P<0.0001. FIG. 10B. Both cytosolic and nuclear fractions were prepared from HEY and OVCAR8 cells. Western blotting was performed with these fractions to detect REL. α-Tubulin was used as cytosolic marker and HDAC1 used as nuclear marker.

### DETAILED DESCRIPTION

### Definitions

Unless defined otherwise, all technical and scientific terms used herein have the same meaning as commonly understood in the art to which this invention pertains and at the time of its filing. Although various methods and materials similar or equivalent to those described herein can be used in the practice or testing of the present invention, suitable methods and materials are described below. However, the person of ordinary skill in the art should understand that the methods and materials used and described are examples and may not be the only ones suitable for use in the invention. Moreover, it should be understood that as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by the person of ordinary skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

As used herein and in the appended claims, the singular forms “a,” “an,” and “the” include plural references unless specifically stated otherwise.

As used herein, the term “about” means approximately, roughly, around, or in the region of, and unless otherwise indicated, it refer to a value that is no more than 10% above or below the value being modified by the term.

As used herein, the term “linked” refers to a covalent linkage.

As used herein, the term “FAP” refers to fibroblast activation protein-α. The human FAP gene is located on chromosome 2q23 with the length of approximately 73 kb and contains 26 exons. FAP is a 97-kDa type II transmembrane serine proteinase, which is 760 amino acids long with residues 1-4 of the intracellular domain. 5-25 of the transmembrane domain and 26-760 of the extracellular domain (Lee et al., 2006). In addition, FAP has five presumptive N-linked glycosylation sites at asparagine residues 49, 92, 227, 314 and 679, of which glycosylation seems necessary for FAP endopeptidase activity (Sun et al., 2002). FAP belongs to the prolyl peptidase family, which also includes dipeptidyl peptidase IV (DPPIV, CD26), DPP7 (DPP II, quiescent cell proline dipeptidase), DPP8, DPP9, and prolyl carboxypeptidase (PCP, angiotensinase C) (Fitzgerald and Weiner, 2020). These proteins contain a catalytic triad of serine, aspartic acid and histidine (Rosenblum and Kozarich, 2003). The serine acts as a nucleophile, cleaving N-terminal Pro-X peptide bonds, where X is any amino acid except proline or hydroxyproline. Like other members of the family, FAP has dipeptidyl peptidase enzymatic activity, but it also has endopeptidase activity, sometimes referred to as gelatinase activity. FAP's endopeptidase activity prefers amino acid sequences of Gly-Pro-X, is most effective where X is Phe or Met, and least effective when X is His or Glu (Collins et al., 2004). Furthermore, FAP is ineffective with large charged amino acids at position P4 and P2′ (Edosada et al., 2006; Aggarwal et al., 2008; Huang et al., 2011). In many reports, FAP is known to be oncogenic, and although its biological role is suggested to be enzymatic activity-dependent, there are some evidence showing that its enzymatic activity is unnecessary for tumorigenicity (Ramirez-Montagut et al., 2004; Huang et al., 2011; Lv et al., 2016)

As used herein, the term “siRNA” (small interfering RNA, also known as short interfering RNA or silencing RNA) is a non-coding double-stranded RNA, and is usually between 20 and 24 base pairs in length. siRNAs are highly specific and usually synthesized to complement and match the protein-encoding nucleotide sequence of the target mRNA to reduce the translation of the target mRNAs (mRNAs) and the synthesis of the protein. After entry into the cytoplasm, siRNA is loaded onto multiprotein RNA-inducing silencing complex (RISC) directly, and then the duplex RNA is unwound leaving the anti-sense strand to guide RISC to complementary mRNA for subsequent endonucleolytic cleavage, thereby commencing the RNA interference process by base-pairing with target mRNA for the mRNA degradation.

As used herein, the term “knock down (KD)” or “silencing” refers to a significant reduction of FAP mRNA or FAP protein (10% or more reduction). Thus, the term “anti-FAP” refers to knock down or silencing of FAP activity.

As used herein, the term “EpCAM” refers to the epithelial cell adhesion molecule (CD326) that is a glycoprotein of ˜40 kd. EpCAM was originally identified as a marker for epithelial origin carcinoma due to its high expression on rapidly proliferating cancer cells. EpCAM is a member of CAM (cell adhesion molecule) family, which includes four archetypal CAM families: cadherins, selectins, integrins, and immunoglobulin CAM (Ig-CAM) superfamily (Trzpis et al., 2007). However, there exist several CAMs that do not belong to any of the four CAM families, and epCAM is the most prominent example. EpCAM has (1) an extracellular domain starting with a signal sequence followed by an epidermal growth factor-like repeat, a human thyroglobulin repeat (TY), and a cysteine-poor domain; (2) a single-spanning transmembrane domain; and (3) an intracellular domain containing an NPXY internalization motif and several α-actinin binding sites (Baeuerle and Gires, 2007; Balzar et al, 1999). Some of the functions ascribed to EpCAM are cell-cell adhesion, cell migration, metastasis, differentiation, proliferation, morphogenesis (organogenesis and regeneration), signaling, reorganization of the actin cytoskeleton, cell cycle, metabolism, etc. (Okegawa et al., 2004).

As used herein, the term “aptamer” refers to an oligonucleotide molecule that binds to a target protein. Aptamers can be RNA or DNA molecule. The embodiments described herein are typically RNA-based aptamers, and it will be understood that the nucleotides of any thymine in the aptamer nucleotide sequence are presented as uracil. The term “EpCAM aptamer” refers to an aptamer that can bind to an extracellular domain of EpCAM protein on the cell surface.

As used herein, the terms “bind,” “binding” refer to any type of any interaction, whether direct or indirect, that affects the specified receptor or receptor subunit, which includes but is not limited to covalent binding, hydrogen binding, electrostatic binding, biological tethers, transmembrane attachment, cell surface attachment and expression. The terms “specifically bind,” “specific binding,” refer to an adherence of one molecule for another which reflects a complementarity between them, for example a ligand and receptor, an enzyme and substrate, an antibody and antigen or hapten, aptamer and target, or two complementary nucleotide strands.

As used herein, the term “gene expression” refers to a process by which information from a gene is used to synthesize of a functional gene product. A gene product is often a protein, but in a non-protein coding gene such as transfer RNA (tRNA) or small nuclear RNA (snRNA) gene, the product is a functional RNA.

As used herein, the term “gene therapy” refers to the purposeful delivery of genetic material to cells for the purpose of treating disease or biomedical investigation and research. Gene therapy includes the delivery of a polynucleotide to a cell in order to suppress, silence, augment, or alter expression of an endogenous nucleotide sequence, or to produce a specific biologically active molecule not naturally synthesized by the cell. In some cases, the polynucleotide itself, when delivered to a cell, can alter the expression of a specific gene in the cell.

As used herein, the term “nucleotide” refers to a compound consisting of a nucleoside linked to a phosphate group, which forms the basic structural unit of nucleic acid polymers such as deoxyribonucleic acid (DNA) and ribonucleic acid (RNA), and unless otherwise limited, encompasses known analogs having the essential nature of natural nucleotides in that they hybridize to their complementary nucleotides.

As used herein, the terms “oligonucleotide” and “polynucleotide” refer to oligomers and polymers, respectively, of natural and/or modified nucleotides, including deoxyribonucleic acid, ribonucleic acid, substituted and alpha-anomeric forms thereof, peptide nucleic acids (PNA), locked nucleic acids (LNA), phosphorothioate, methylphosphonate, and the like. In addition, unless otherwise indicated, the terms refer to the specified sequence as well as the complementary sequence thereof. Moreover, DNAs or RNAs comprising unusual bases, such as inosine, or modified bases, such as tritylated bases, to name just two non-limiting examples, are oligonucleotides or polynucleotides as the term is used herein. Thus, the term oligonucleotide or polynucleotide as it is employed herein encompasses such chemically, enzymatically or metabolically modified forms of oligonucleotides or polynucleotides, as well as the chemical forms of DNA and RNA characteristic of viruses and cells, including inter alia, simple and complex cells. A great variety of modifications have been made to DNA and RNA that serve many useful purposes known to those of skill in the art.

As used herein, the term “complementary” refers to a nucleotide sequence, usually an oligomer of at least 8-10 nucleotides, that aligns in an antiparallel manner with Watson-Crick base pairing at all positions with a target sequence for a contiguous length of nucleotides of at least 8-10 base pairs. Such a complementary sequence binds to the target sequence under stringent conditions. The term “substantially complementary” refers to a nucleotide sequence, usually an oligomer, that aligns in an antiparallel manner with Watson-Crick base pairing at sufficient positions with a target sequence to bind the target sequence under stringent conditions. A substantially complementary sequence is 80% homologous to a fully complementary sequence, preferably 85% c complementary, more preferably 90% complementary or 95% complementary, and most preferably 98% complementary or 99% complementary. A substantially complementary sequence can have one, two, or three non-complementary bases in the sequence, for example.

As used herein, the term “target nucleic acid” encompasses DNA, RNA (comprising pre-mRNA and mRNA) transcribed from such DNA, and also cDNA derived from such RNA, coding, noncoding sequences, sense or antisense polynucleotides. The complementary bonding (hybridization) of an oligomeric compound with its target nucleic acid interferes with the normal function of the nucleic acid.

As used herein, the term “cancer” or “tumor” refers to any neoplastic abnormal uncontrolled cell growth in a patient, including an initial tumor and any metastases. In some embodiments, cancer refers to a localized overgrowth of cells that has not spread to other parts of a subject, i.e., a benign tumor. In other embodiments, cancer refers to a malignant tumor, which has invaded and destroyed neighboring body structures and spread to distant sites. the term “cancer cells” refers to the cells that acquire characteristics of cancer during their development, such as aberrant cell growth due to abnormally increased cell proliferation or abnormally reduced apoptosis, and lack of contact inhibition of growth in vitro, and includes tumor cells that is either incapable of or capable of metastasis in vivo

As used herein, the term “cancer marker” refers to anything, in particular anything produced by cancer cells and presented on the cell surface or membrane of cancer cells to provide information about a cancer, such as its aggressiveness, possibility of treatment with a targeted therapy, its responsiveness to the treatment, etc.

As used herein, the term “cancer susceptible to FAP knock down or silencing” refers to a cancer wherein the aggressiveness, malignancy and/or viability is reduced as a result of FAP knock down or silencing compared with non-FAP knock down or silencing cancers, includes ovarian cancer.

As used herein, the term “subject” refers to an animal or a human individual, which is the object of treatment, diagnosis, observation or experiment. The term includes any animal, preferably a mammal, and in particular human patients, in particular human cancer patients or humans in need of treatment for cancer, including ovarian cancer.

As used herein, a subject “in need” refers to a subject that is suspected of having, has been diagnosed as having, or is at risk of developing a disease that can be ameliorated, treated or prevented by administration of the pharmaceutical composition of this invention.

As used herein, the terms “treating,” and “treatment” refer to providing any type of medical management to a subject. Treating or treatment therefore includes, but is not limited to, administering a pharmaceutical composition comprising the invention to a subject using any known method for purposes such as curing, reversing, alleviating, reducing the severity of, inhibiting the progression of, or reducing the likelihood of a disease, disorder, or condition or any of its symptoms, and also includes reduction or elimination of one or more symptoms or manifestations of a discase, disorder or condition.

As used herein, the term “pharmaceutical composition” as part of the disclosed invention, refers to a product comprising one or more of the disclosed oligonucleotides comprising anti-FAP siRNA or anti-FAP siRNA linked to EpCAM aptamers, and an optional carrier comprising inert ingredient(s), as well as any product that results, directly or indirectly, from combination, complexation or aggregation of any two or more of the ingredients, or from dissociation of one or more of the ingredients, or from other types of reactions or interactions of one or more of the ingredients. Pharmaceutical composition embodiments can be in the form of a solid, liquid or gas (aerosol).

As used herein, the terms “administer,” and “administration,” when used with respect to an agent, mean providing the agent to a subject using any of the various methods or delivery systems for administering agents or pharmaceutical compositions known to those skilled in the art. Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection, directly into the lumen of the bladder, directly into the tumor, or infusion techniques. In a specific embodiment, the compositions are administered parenterally. Pharmaceutical compositions can take the form of one or more dosage units, e.g., a tablet or a capsule.

As used herein, the term “pharmaceutical formulation” refers to a composition containing an active agent and a carrier mixture, combined to produce a medicinal product, such as a sterile solution, a capsule, a tablet, a powder, a granule, a solution, an emulsion, a gel, ointment, cream, lotion or the like for administration to a subject by any convenient method. A “pharmaceutical formulation” also includes delayed-release or sustained-release preparations. The medicinal product will vary by the route of administration which is to be used. For example, oral drugs are normally taken as tablet or capsules. Preferred pharmaceutical formulations for embodiments of the invention include forms of injection such as intravenous, intraarterial, and intraventricular or intracranial injection or infusion into the brain or CSF.

As used herein, the term “pharmaceutically acceptable carrier” refers to any carrier(s), used to facilitate administration of a pharmaceutical compound of the invention, which do not induce damage to the compound or to a subject receiving the pharmaceutical compound, is nontoxic and non-reactive, and is chemically and biologically compatible with the active agent. For example, pharmaceutically acceptable carriers may be large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Therefore, the term “pharmaceutically acceptable carrier” refers to nontoxic and nonreactive compound or agent that facilitates the incorporation of an active agent or pharmaceutical compound to form a pharmaceutical composition for administration to a subject.

As used herein, the term “cancer therapy” refers to any method(s), useful for the treatment of a cancer or one or more symptoms thereof. In certain embodiments, the terms “therapy” and “therapies” refer to chemotherapy and/or radiation therapy, radioimmunotherapy, hormonal therapy, targeted therapy, toxin therapy, pro-drug activating enzyme therapy, protein therapy, antibody therapy, small molecule therapy, epigenetic therapy, demethylation therapy, histone deacetylase inhibitor therapy, differentiation therapy, antiangiogenic therapy, biological therapy including immunotherapy and/or other therapies useful in the treatment of a cancer or one or more symptoms thereof. In a specific embodiment, a therapy is administration of an effective

As used herein, an “adjunct cancer therapeutic agent” pertains to an agent that possesses selectively cytotoxic or cytostatic effects to cancer cells over normal cells. Adjunct cancer therapeutic agents may be co-administered with an oligonucleotides comprising anti-FAP siRNA linked to EpCAM aptamers.

As used herein, the term “adjunct cancer therapy protocol” refers to a therapy, such as surgery, radiation therapy, chemotherapy, gene therapy. DNA therapy, adjuvant therapy, neoadjuvant therapy, RNA therapy, DNA therapy, viral therapy, immunotherapy, laser therapy, nanotherapy or a combination thereof, and may provide a beneficial effect when administered in conjunction with administration of an oligonucleotides comprising anti-FAP siRNA linked to EpCAM aptamers. Such beneficial effects include reducing tumor size, slowing rate of tumor growth, inhibiting metastasis, or otherwise improving overall clinical condition, without necessarily eradicating the cancer. Cytostatic and cytotoxic agents that target the cancer cells are specifically contemplated for combination therapy. Likewise, agents that target angiogenesis or lymphangiogenesis are specifically contemplated for combination therapy.

As used herein, the term “therapeutically effective amount” in the context of cancer treatment refers to an amount sufficient to reduce expression of the targeted FAP gene or FAP protein (including by reducing transcription or translation of the gene or mRNA, respectively), and/or an amount sufficient to reduce the severity and duration of cancer, ameliorate one or more symptoms of cancer, prevent the advancement of cancer, cause regression of cancer, and/or enhance or improve the therapeutic effect(s) of another therapy. Typically, an effective amount is provided according to a regimen.

### Overview

Disclosed herein are embodiments of an anti-FAP siRNA and oligonucleotides comprising anti-FAP siRNA linked to EpCAM aptamers. To this end, it was demonstrated that FAP is upregulated in advanced stages of ovarian cancer and invasive ovarian cancer cells, that FAP is involved in ovarian cancer cell survival independent of its proteinase function, that the presence of FAP is essential for NF-κB and BIRC5 (survivin) activity and PRKDC localization in lipid rafts for the survival of ovarian cancer cells, and that anti-FAP siRNA effectively suppresses intraperitoneal ovarian cancer development in vivo mice model.

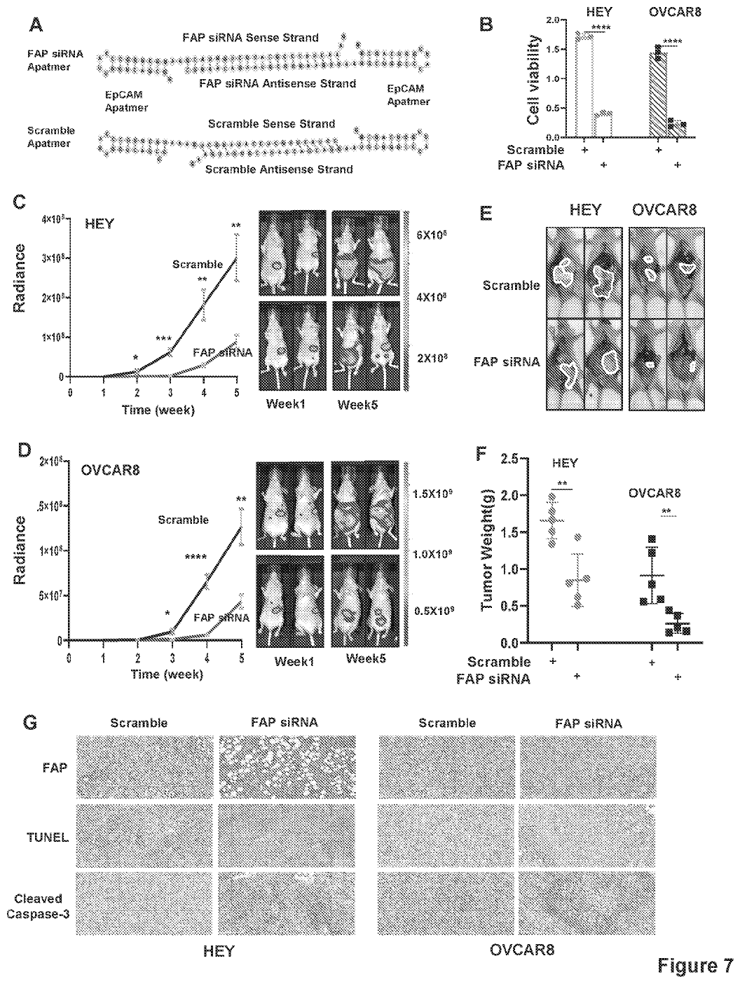

In addition, in some embodiments of this invention (FIG. 7), for ovarian cancer targeted delivery of anti-FAP siRNA, each of the sense oligonucleotide and antisense oligonucleotide of anti-FAP siRNA is linked at one end to an aptamer RNA of EpCAM, in order to form an aptamer-ds-siRNA-aptamer structure (FIG. 7A) for effective cancer surface antigen recognition and expedited internalization of the siRNA. Furthermore, each the sense oligonucleotide and antisense oligonucleotide of anti-FAP siRNA linked to an aptamer RNA of EpCAM is transcribed in vitro in the presence of 2′-F-deoxyguanosine or natural deoxyguanosine to enhance the stability of the RNA strands.

Ovarian cancer is the deadliest gynecological cancer, and its high mortality rate is mainly due to the lack of symptom at early stage. Current mainstay of ovarian cancer therapy is the combination of surgery and chemotherapy. However, majority of patients will develop therapeutic resistance, suffer relapse and eventually succumb to the discase. Therefore, in order to tackle such unmet need to treat ovarian cancer and find genes upregulated in advanced ovarian cancer, we performed the analysis of the Cancer Genome Atlas (TCGA) ovarian cancer data set, and discovered FAP (fibroblast activation protein-α) is one of those upregulated proteins. Thus, in this invention we provide anti-FAP siRNAs, in particular anti-FAP siRNAs linked to aptamers that recognize and bind to a cancer marker protein, in particular to epithelial cell adhesion molecule (EpCAM).

FAP is a 97-kDa type II transmembrane serine proteinase and a member of the prolyl peptidase family, which also includes dipeptidyl peptidase IV (DPPIV, CD26), DPP7 (DPP II, quiescent cell proline dipeptidase), DPP8, DPP9, and prolyl carboxypeptidase (PCP, angiotensinase C). Like many other members of the family. FAP has dipeptidyl peptidase enzymatic activity, but FAP also has endopeptidase activity (gelatinase activity) (Fitzgerald and Weiner, 2020; Goldstein et al.; 1997; Rosenblum. 2003; Collins et al., 2004; Edosada et al, 2006; Aggarwal et al. 2008; Huang et al, 2011).

FAP is usually absent in most normal adult tissues, but FAP expression has been reported to be elevated under pathological conditions such as fibrosis, arthritis and cancer, especially in tumor stroma (cancer-associated fibroblasts). Actually, elevated FAP expression and activation were shown in various cancers such as adenocarcinoma of the colon and stomach, invasive ductal carcinoma of the breast, and malignant melanoma, and it is suggested that FAP may affect clinical outcomes via its effects on extracellular matrix remodeling, intracellular signaling regulation, angiogenesis, epithelial-to-mesenchymal transition and immunosuppression (Fitzgerald and Weiner, 2020).

FAP has also been reported to be present in almost all ovarian cancer, but not in normal ovary tissue and benign ovarian tissues. FAP expression in ovary cancer stroma is certain, and its expression was also detected in ovarian cancer cell lines.

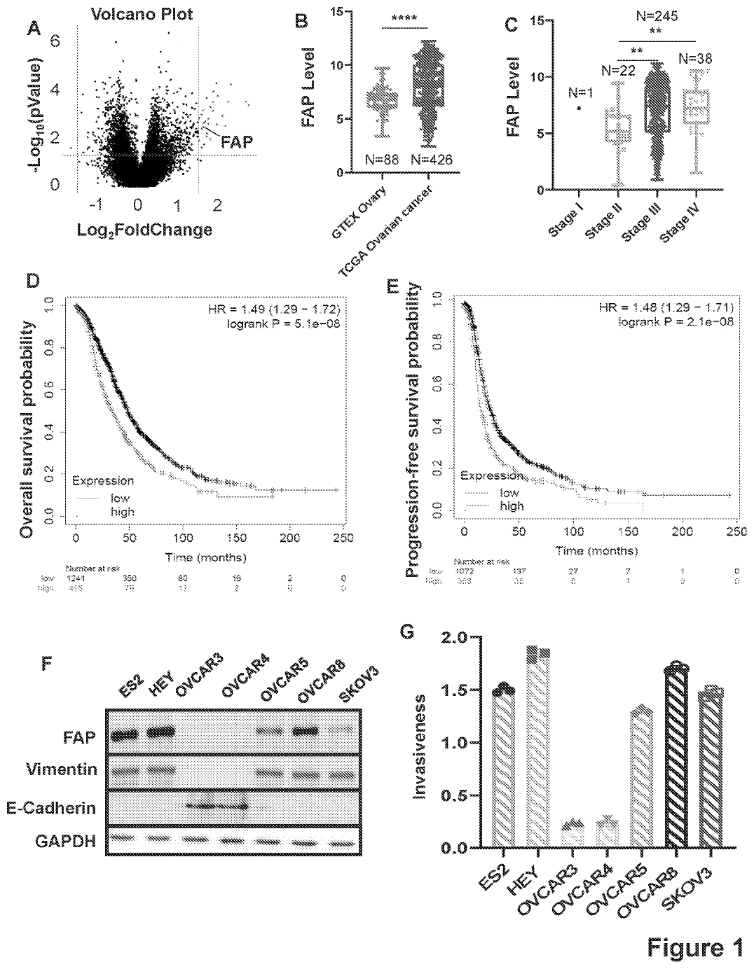

In an embodiment, to identify candidate genes critical for ovarian cancer progression, a volcano plot was generated using TCGA ovarian cancer data set and discovered that total of 24 genes were upregulated in advanced stages of patients (FIG. 1A and Table 5). FAP was one of them, and specific attention was paid to FAP because it is a proteinase and regarded as targetable molecule. As a next step, FAP's relevance to ovarian cancer progression was investigated by comparing its expression between normal ovary tissue and ovarian cancer and found that FAP was much higher in ovarian cancer (FIG. 1B). Further analysis also showed that FAP expression is higher in Stage III and IV patients than Stage II patients (FIG. 1C) and that FAP was inversely correlated with patient survival (FIGS. 1D and 1E).

In another embodiment, by using Matrigel and western blot with antibodies against E-cadherin (an epithelial marker), vimentin (a mesenchymal marker), and FAP protein, it was shown that vimentin-expressing mesenchymal-like cell lines were highly invasive while E-cadherin-expressing epithelial-like cell lines barely invaded Matrigel (FIG. 1F and FIG. 1G), and that FAP was only detected in mesenchymal-like invasive ovarian cancer cell lines (FIG. 1F), indicating that FAP is specifically involved in tumorigenic events in mesenchymal-like ovarian cancer.

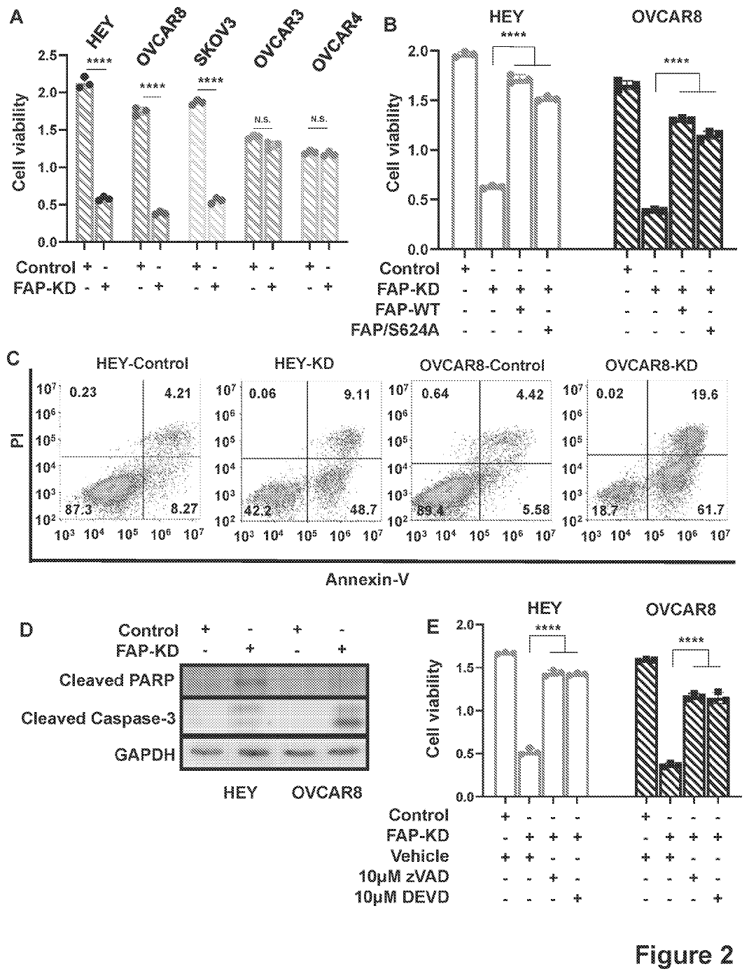

In another embodiment, to investigate the effect of FAP silencing on ovarian cancer behaviors, a set of commercially available anti-FAP shRNAs were screened in HEY cells, which is a human ovarian carcinoma cell line derived from a human ovarian cancer xenograft (HX-62) originally grown from a peritoneal deposit of a patient with moderately differentiated papillary cystadenocarcinoma of the ovary, and were able to identify only one effectively depleting anti-FAP shRNA. When the cells were lentivirally transduced with this shRNA, it led to massive cell death in FAP-expressing invasive ovarian cancer cell lines while the survival of non-invasive lines (FAP-negative) was not affected (FIG. 2A).

Experimental evidence from both in vitro and in vivo studies showed that the proteinase activity of FAP may be a key contributor to its cancer-promoting role though there are other reports indicating that the proteinase activity of FAP may not play a critical role in cancer cell growth. Here, we also showed that a proteinase-inactivated FAP behaved similarly to wild-type FAP in ovarian cancer cell growth, proving that the enzymatic role of FAP is not essential for cancer promotion.

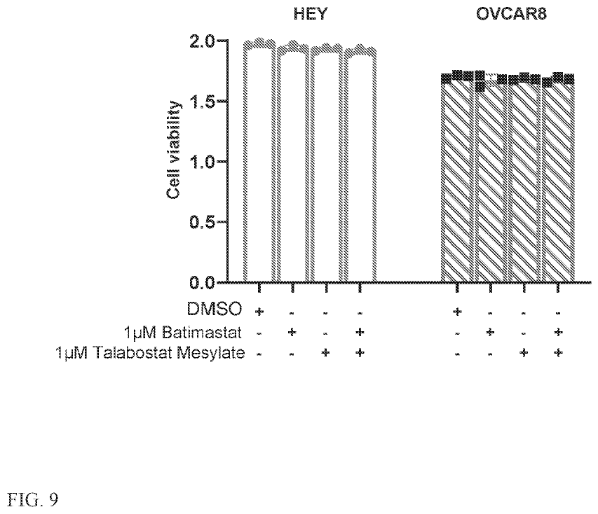

In another embodiment, to determine if FAP's proteinase function is important for cell survival, wild-type or proteinase-inactive mutant of FAP was introduced into FAP-knockdown cells, and with cell viability assay (CCK8) showed that both similarly abrogated cell death trigged by FAP-knockdown by anti-FAP shRNA (FIG. 2A). These results indicate that proteinase activity is dispensable for the role of FAP in cell survival. This notion is further supported by another embodiment showing that metalloprotease inhibitor Batimastat and dipeptidase inhibitor Alabostat either alone or together displayed little effect on survival of HEY and OVCAR8 cells (FIG. 9).

However, in spite of the fact that FAP expression seems associated with cancer cell survival, the underlying biological mechanisms remain unclear. To elucidate this matter, in some embodiments of this invention, a few signal transduction pathways blocked by FAP suppression were explored.

In another embodiment, it was shown FAP-knockdown triggered apoptosis, by performing Annexin V/propidium iodine (PI) staining-based flow cytometry in control and FAP-knockdown HEY and OVCAR8 cells (FIG. 2C). As a result, western blot showed cleaved PARP and cleaved caspase-3 in FAP-knockdown cells (FIG. 2D). Moreover, in another embodiment, it was shown that both pan-caspase inhibitor zVAD and caspase-3 inhibitor DEVD blocked anti-FAP shRNA-led cell death (FIG. 2E). Collectively, these results suggest that silencing FAP results in apoptosis of invasive ovarian cancer cells.

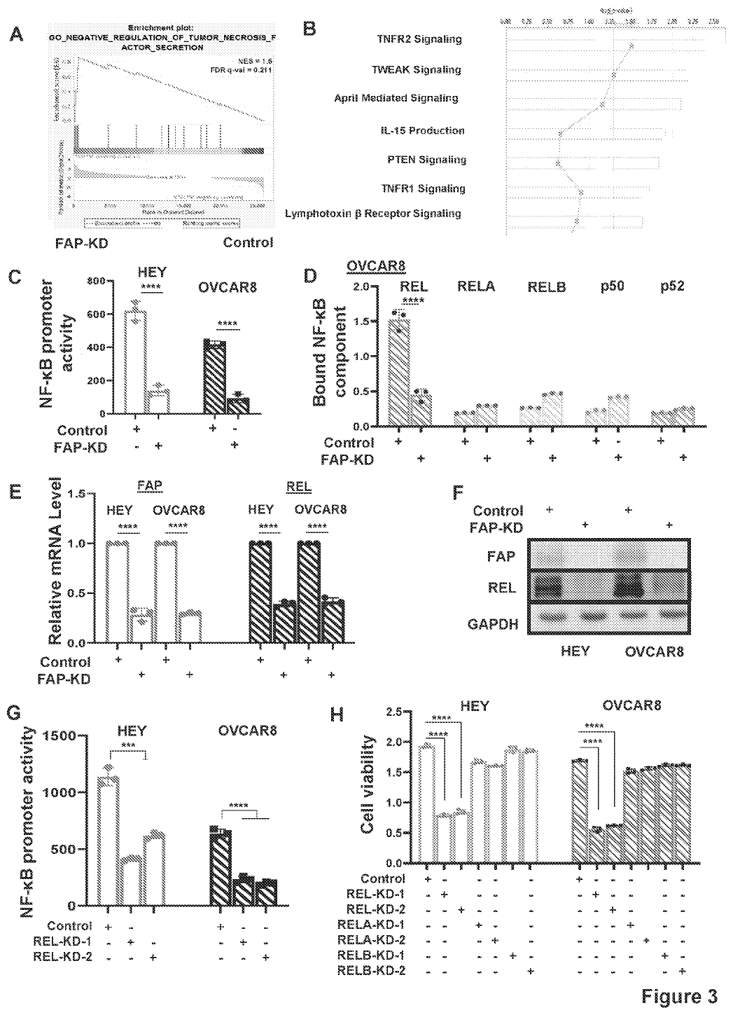

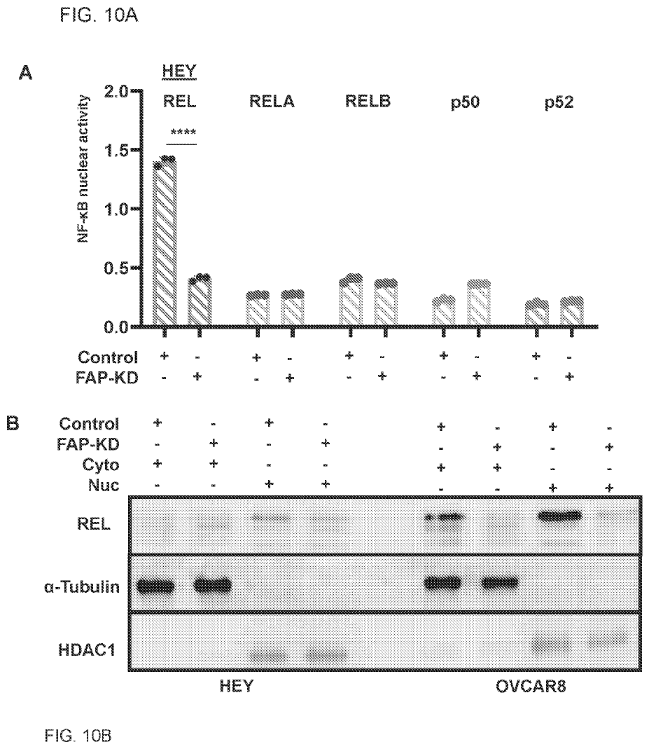

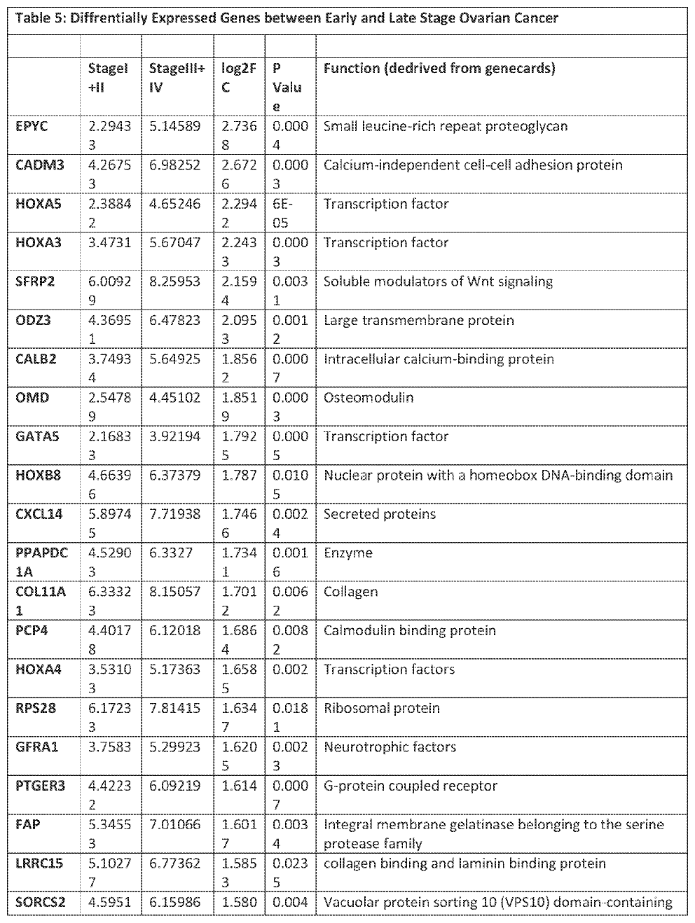

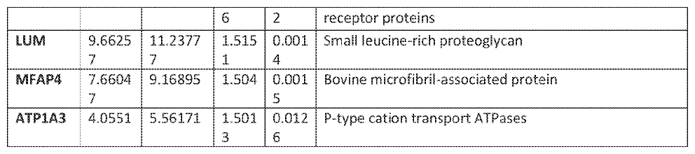

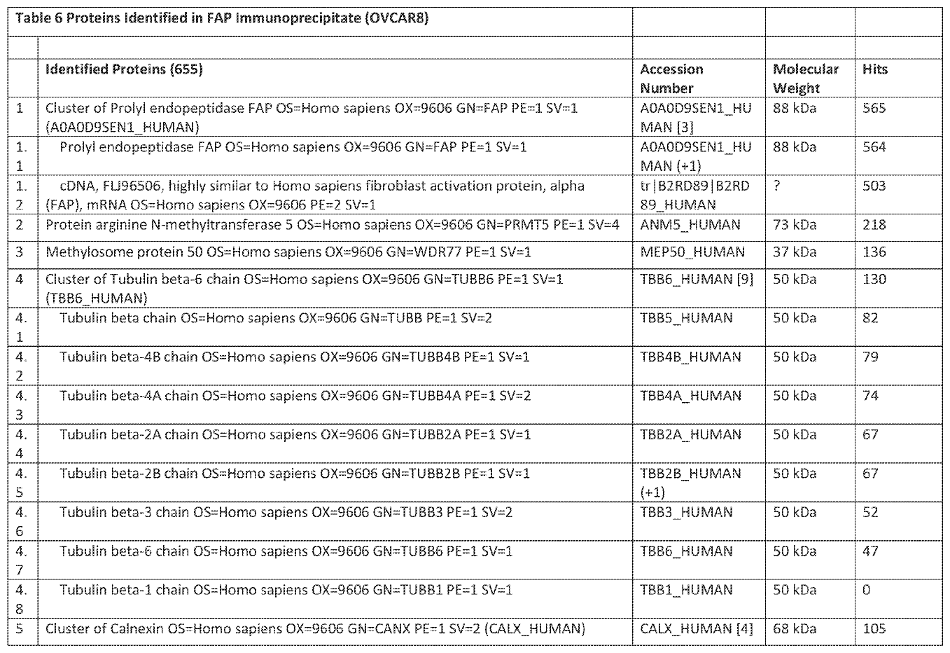

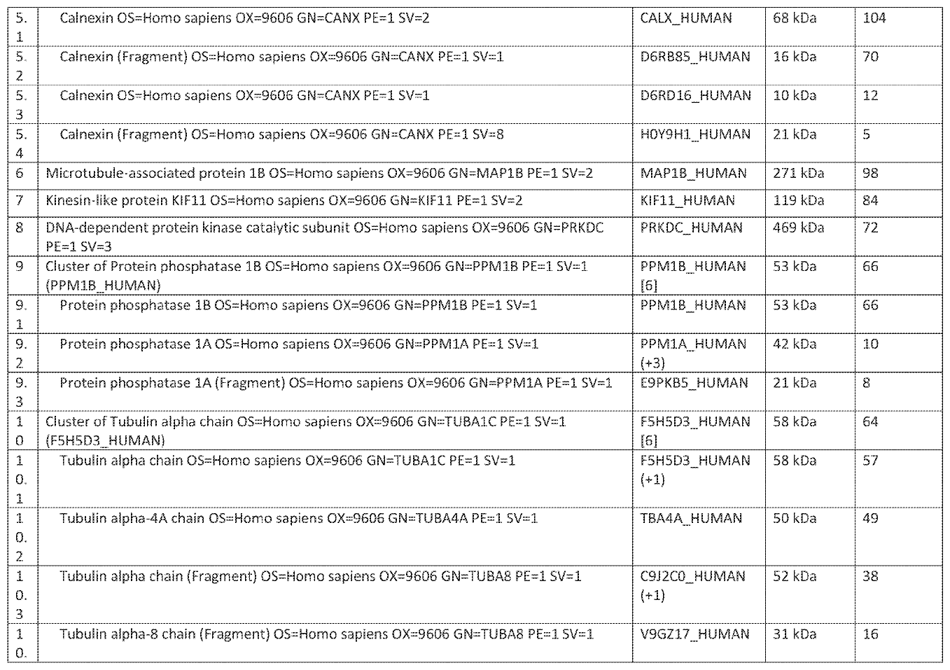

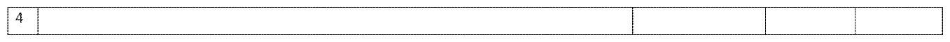

As a result of RNAseq from both control and FAP-knockdown OVCAR8 cells (a human ovarian carcinoma cell line) and Gene Sequence Enrichment Analysis (GSEA) and Ingenuity Pathway Analysis (IPA), tumor necrosis factor receptor (TNFR) signaling, whose signaling is well established to promote cell survival by promoting NF-κB activity, was shown impaired in FAP-knockdown cells (FIGS. 3A and 3B). In addition, when NF-κB activity was measured by using luciferase activity, we detected over 80% reduction in NF-κB activity in FAP-knockdown cells in comparison with their respective controls (FIG. 3C). Further ELISA-based NF-κB transactivation assay showed marked reduction of REL binding to oligonucleotides containing NF-κB consensus sequence in FAP-knockdown cells (FIG. 3D and FIG. 10A) due to dramatic reduction of both REL mRNA and protein in FAP-knockdown cells (FIGS. 3E and 3F). In addition, it was shown that silencing REL exhibited similar inhibitory effect in NF-κB activity to that of FAP knockdown (FIG. 3G), and that only silencing REL (but not RELA or RELB) was able to decrease cell viability (FIG. 3H). These results suggest that FAP is essential for the survival of invasive ovarian cancer cells by sustaining REL-associated NF-κB activity.

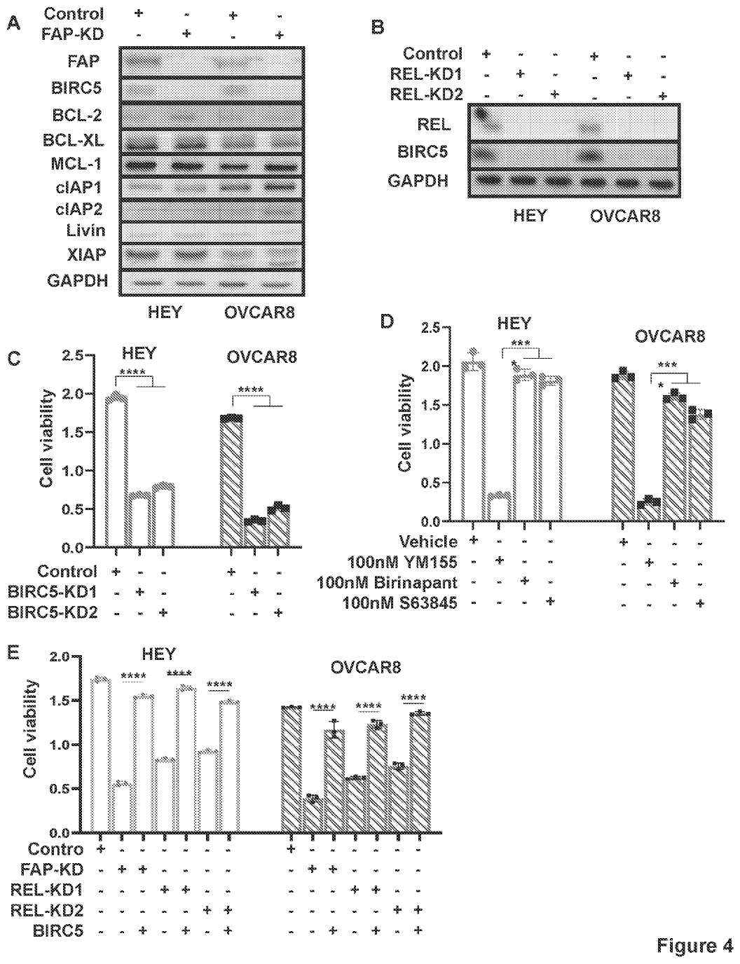

NF-κB signaling promotes cell survival by maintaining the expression of various pro-survival proteins (LaCasse et al., 1998), and thus in some embodiments, we examined the levels of known NF-κB-regulated pro-survival proteins in control and FAP-knockdown cells. Knockdown of FAP and REL diminished BIRC5 (FIG. 4A and FIG. 4B), and knockdown or inhibition of BIRC5 evidently reduced cell number (FIG. 4C and FIG. 4D). When BIRC5 was ectopically expressed in HEY or OVACR8 cells, the cell death inducing-effect of FAP shRNA or REL shRNA was lost, suggesting that FAP-REL signaling axis promotes ovarian cancer cell survival by sustaining BIRC5 expression.

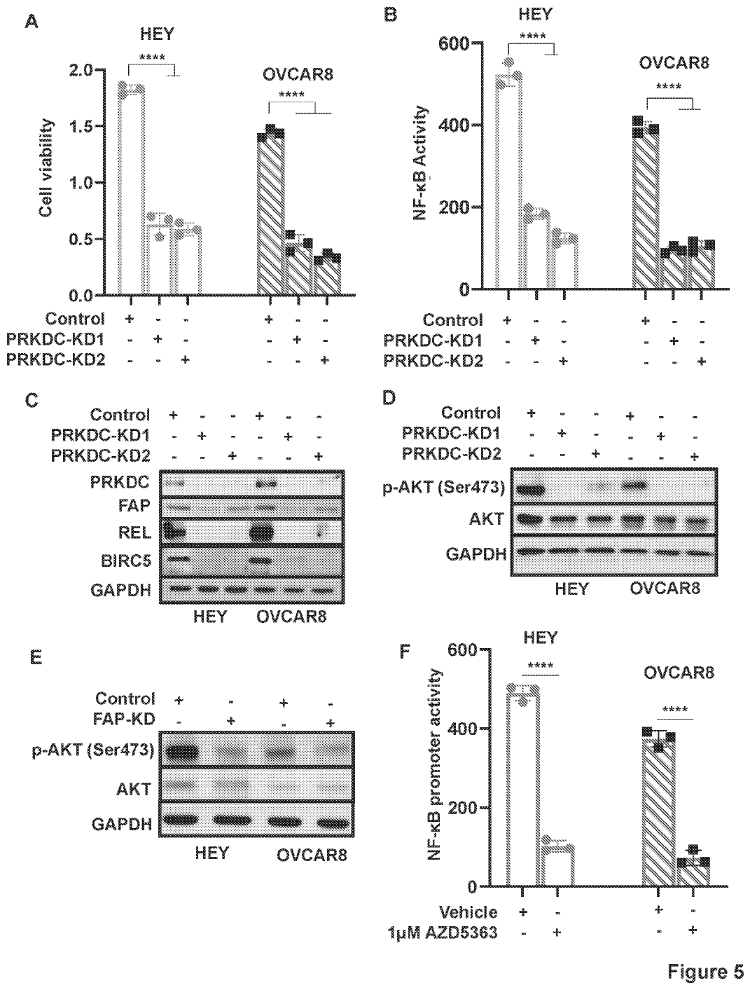

Further, in another embodiment, we identified PRKDC (Table 6), which has been reported to regulate NF-κB activity (Wei et al, 2013; Basu et al., 1998), as one of FAP interacting proteins, and discovered that knockdown of PRKDC with shRNAs reduced cell viability (FIGS. 5A and S5A) as well as NF-κB activity (FIGS. 5B and S5B), and abolished REL and BIRC5 but much less effect on FAP (FIG. 5C).

Since PRKDC has been shown to promote cell survival involving AKT phosphorylation (Lu et al., 2006; Bozulic et al., 2008) while Akt signaling pathway is well characterized for its importance in NF-κB activation (Ozes et al., 1999; Madrid et al., 2001), in another embodiments, we analyzed Akt phosphorylation in control- and PRKDC-knockdown HEY and OVCAR8 cells to show that the level of Akt phosphorylation was almost completely abolished in PRKDC-knockdown cells (FIG. 5D). Similarly, silencing FAP also greatly lowered the abundance of phosphorylated Akt (FIG. 5E). As Akt inhibitor diminished NF-κB activity in both HEY and OVCAR8 cells (FIG. 5F), these results support the notion that PRKDC acts as a function connection between FAP and NF-κB activation in ovarian cancer cells.

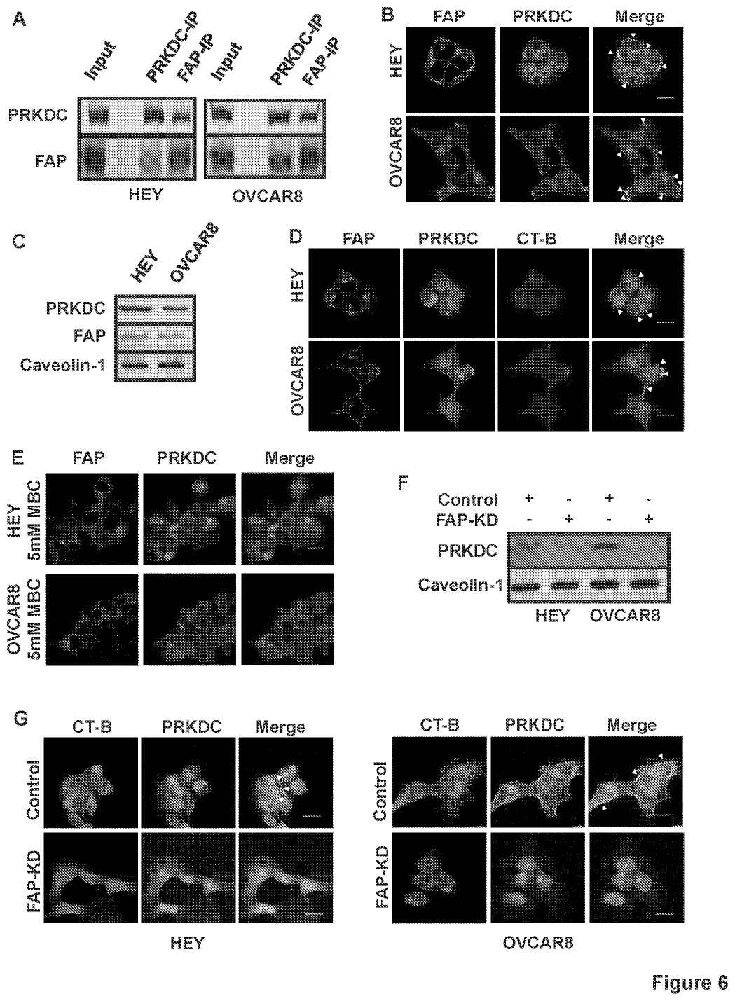

In another embodiments, we also showed that FAP directly interacts with PRKDC in lipid rafts by co-immunoprecipitation confocal immunofluorescence staining (FIGS. 6A, 6B, 6C and 6D), and demonstrated that FAP recruits PRKDC to lipid raft to launch its signaling for NF-κB activation in ovarian cancer cells.

Because of such FAP's tumor-specific expression and tumor-promoting capability, efforts have been exerted to develop small molecular inhibitors targeting FAP activity and monoclonal antibodies targeting its extracellular domain. Unfortunately, clinic trials with these agents have not been unsuccessful and the underlying reasons have not been addressed. Therefore, there is a need for a new therapeutic approach to silence/suppress FAP gene and/or protein to cure cancers, in particular ovarian cancer, and anti-FAP siRNA can be one of such approaches.

To address this matter, in another embodiment, an oligonucleotide was prepared comprising an ani-FAP siRNA linked to an aptamer of EpCAM for siRNA delivery. EpCAM is highly expressed in ovarian cancer but not in other tissues in peritoneal cavity. The epithelial cell adhesion molecule (EpCAM. CD326) is a glycoprotein of ˜40 kd, and non-cancerous normal epithelial cells generally express EpCAM at a lower level than cancer cells (Trzpis et al., 2007). EpCAM was originally identified as a marker for carcinoma due to its high expression on rapidly proliferating cancers, especially on tumors of epithelial origin. In early studies, the role of EpCAM was suggested to be a cell-cell adhesion molecule. However, many other recent studies proposed the roles for EpCAM not only in cell adhesion but also in diverse processes such as cell signaling, migration, proliferation, and differentiation (Litvinov et al., 1997; Cirulli et al, 1998; Nochi et al., 2004; Osta et al., 2004). Further, there have been the implications of the newly identified functions of EpCAM in view of its relevance in carcinoma of epithelial origin. However, it was also discovered that EpCAM aptamer can be used for mesenchymal-origin ovarian cancer cells by showing that an oligonucleotide comprising an anti-FAP siRNA linked to EpCAM aptamers effectively suppressed mesenchymal-like ovarian cancer development.

In an embodiment, for ovarian cancer specific delivery of anti-FAP siRNA, a sense strand template oligonucleotide (SEQ ID NO:34) and an antisense strand template oligonucleotide (SEQ ID NO:35) of anti-FAP siRNA was designed as well as ds-scrambled control RNA (SEQ ID NOs:32 and 33) for T7 polymerase in vitro transcription in the presence of 2′-fluoro (F)-deoxycytidine and deoxyguanosine, to create a sense strand linked at its 5′-end to an EpCAM RNA aptamer and an antisense strand linked at its 5′-ends to an EpCAM RNA aptamer, and consequently to create a ds-siRNA both ends of which are EpCAM aptamers (FIG. 7A). As a result of treatment of human ovarian cancer cells with an oligonucleotide of aptamer-control (scrambled sequence)-aptamer or aptamer-anti-FAP siRNA-aptamer, it was found that the oligonucleotides of aptamer-anti-FAP siRNA-aptamer reduced approximately 80% of cell viability compared with the aptamer-control-aptamer (FIG. 7B). In another embodiment, it was also shown that intraperitoneal xenograft development by human ovarian cancer cells in female athymic nude mice were much smaller in mice administered with the oligonucleotides of aptamer-anti-FAP siRNA-aptamer than in that in mice administered with the nucleotides of aptamer-scrambled (control)-aptamer (FIGS. 7C and 7D). These results provide pre-clinical evidence that the nucleotides comprising anti-FAP siRNA linked to EpCAM aptamers can potentially be an effective therapeutic tool against ovarian cancer.

In certain embodiments, provided is an anti-FAP siRNA as well as 4 types of FAP-silencing oligonucleotides which comprise anti-FAP siRNA and EpCAM aptamers for FAP gene knock down/silencing.

(1) EpCAM aptamer:
(SEQ ID NO: 1)
5′-GGCGACUGGUUACCCGGUCGUAA-3′
Reverse nucleotide sequence of
EpCAM aptamer (3′-rematpa-5′):
(SEQ ID NO: 2)
5′-AAUGCUGGCCCAUUGGUCAGCGG-3′
(2) Anti-FAP siRNA
Sense strand:
(SEQ ID NO: 3)
5′-GCAUUGUCUUACGCCCUUCAA AG-3′
Anti-sense strand:
(SEQ ID NO: 4)
5′-UUGAAGGGCGUAAGACAAUGC-3′
(3) FAP silencing oligonucleotide 1
(5′-aptamer-3′-5′-siRNA-3′)
Sense strand:
5′-GGCGACUGGUUACCCGGUCGUAA-3′-5′-GCAUUGUCUUACGCCCU
UCAA AG-3
SEQ ID NO: 5
(5′-GGCGACUGGUUACCCGGUCGUAA-GCAUUGUCUUACGCCCUUCAAA
G-3;)
Anti-sense strand:
5′-GGCGACUGGUUACCCGGUCGUAA-3′-5′-UUGAAGGGCGUAAGACA
AUGC-3′
SEQ ID NO: 6
(5′-GGCGACUGGUUACCCGGUCGUAA-UUGAAGGGCGUAAGACAAUGC-
3′;)
(4) FAP silencing oligonucleotide 2
(3′-rematpa-5′-5′-siRNA-3′)
Sense strand:
3′-AAUGCUGGCCCAUUGGUCAGCGG-5′-5′-GCAUUGUCUUACGCCCU
UCAA AG-3
SEQ ID NO: 7
(5′-AAUGCUGGCCCAUUGGUCAGCGG-GCAUUGUCUUACGCCCUUCAAA
G-3;)
Anti-sense strand:
3′-AAUGCUGGCCCAUUGGUCAGCGG-5′-5′-UUGAAGGGCGUAAGACA
AUGC-3′
SEQ ID NO: 8
(5′-AAUGCUGGCCCAUUGGUCAGCGG-UUGAAGGGCGUAAGACAAUGC-
3′;)
(5) FAP silencing oligonucleotide 3
(5′-siRNA-3′-5′-aptamer-3′-)
Sense strand:
5′-GCAUUGUCUUACGCCCUUCAAAG-3-5′-GGCGACUGGUUACCCGGU
CGUAA-3′
SEQ ID NO: 9
(5′-GCAUUGUCUUACGCCCUUCAA AG-GGCGACUGGUUACCCGGUCGU
AA-3′;)
Anti-sense strand:
5′-UUGAAGGGCGUAAGACAAUGC-3′-5′-GGCGACUGGUUACCCGGUC
GUAA-3′
SEQ ID NO: 10
(5′-UUGAAGGGCGUAAGACAAUGC-GGCGACUGGUUACCCGGUCGUAA-
3′;)
(6) FAP silencing oligonucleotide 4
(5′-siRNA-3′-3′-rematpa-5′)
Sense strand:
5′-GCAUUGUCUUACGCCCUUCAA AG-3-3′-AAUGCUGGCCCAUUGGU
CAGCGG-5′
SEQ ID NO: 11
(5′-GCAUUGUCUUACGCCCUUCAAAG-AAUGCUGGCCCAUUGGUCAGCG
G-3′;)
Anti-sense strand:
5′-UUGAAGGGCGUAAGACAAUGC-3′-3′-AAUGCUGGCCCAUUGGUCA
GCGG-5′
SEQ ID NO: 12
(5′-UUGAAGGGCGUAAGACAAUGC-AAUGCUGGCCCAUUGGUCAGCGG-
5′;)

### EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1. General Methods and Materials

Cell culture and reagents: All cell lines were obtained from ATCC (Manassas, VA) and cultured in DMEM (Fisher, Cat #MT10013CM) supplemented with 10% fetal bovine serum (Fisher, Cat #26-140-079) in a humidified incubator containing 5% CO₂ at 37° C. All transfections were performed using Lipofectamine™ 3000 Transfection Reagent (Thermo Fisher, Cat #L3000015) according to manufacturer's protocol. ZVAD (Cat #S7023), DEVD (Cat #S7312), YM155 (Cat #S1130), Talabostat (PT-100) (Cat #S8455), Birinapant (Cat #S7015), Batimastat (Cat #S7155), AZD5363 (Cat #S8019), and S63845 (Cat #S8383) were obtained from Selleckchem (Houston, TX). Primers were synthesized by Integrated DNA Technologies (Coralville, IA). All shRNA constructs were obtained from Sigma Aldrich (Darmstadt, Germany). Methyl-beta-cyclodextrin was purchased from Thermo Fisher (Cal #377110050).

RNA sequencing and RT-qPCR: Total RNA was extracted using RNeasy Kit (Qiagen, Cat #74004; Germantown, MD) according to manufacturer's protocol. For RNA sequencing, RNA (50 μg per sample) was sent to University of Florida (UF) core and sequencing was done on Illumina NovaSeq6000. Raw RNA sequencing data were uploaded to Gene Expression Omnibu and further processed using STAR-htseqcount-deseq2 packages in galaxy (https://usegalaxy.org/). Generated data were then analyzed using web-based GSEA (https://www.gsea-msigdb.org/gsea) and IPA (https://resources.qiagenbioinformatics.com).

RT-qPCR was performed using SYBR™ Green Master Mix (ThermoFisher, Cat #A25742) following manufacturer's protocol. Primer pairs used are as follows.

TABLE 1

| | RT-qPCR Primer |
|---|---|
| | Nucleotide Sequence |
| SEQ ID NO: 21 | FAP Forward: |
| | 5′-ATGAGCTTCCTCGTCCAATTCA-3′ |
| SEQ ID NO: 22 | FAP Reverse: |
| | 5′-AGACCACCAGAGAGCATATTTTG-3′ |
| SEQ ID NO: 23 | BIRC5 Forward: |
| | 5′-GAGGCTGGCTTCATCCACTG-3′ |
| SEQ ID NO: 24 | BIRC5 Reverse: |
| | 5′-ATGCTCCTCTATCGGGTTGTC-3′ |
| SEQ ID NO: 25 | REL Forward: |
| | 5′-GCAGAGGGGAATGCGTTTTA-G-3′ |
| SEQ ID NO: 26 | REL Reverse: |
| | 5′-AGAAGGGTATGTTCGGTTGTTG-3′ |
| SEQ ID NO: 27 | GAPDH Forward: |
| | 5′-ACAACTTTGGTATCGTGGAAGG-3′ |
| SEQ ID NO: 28 | GAPDH Reverse: |
| | 5′-GCCATCACGCCACAGTTTC-3′ |

Immunoprecipitation and mass spectrometry: Samples for co-immunoprecipitation and mass spectrometry were prepared using Thermo Scientific™ Pierce™ Co-Immunoprecipitation Kit (Cat #PI26149) following manufacturer's protocol. For mass spectrometry, samples were precipitated with anti-FAB polyclonal antibody (R&D systems, Cat #AF3715) and then sent to UF core for analysis on TSQ Altis™ Triple Quadrupole Mass Spectrometer (Thermo Fisher). For co-immunoprecipitation, samples were either precipitated with anti-FAB polyclonal antibody or goat anti-PRKDC polyclonal antibody (Abcam, Cat #ab168854).for 2 h and further with TrueBlot™ anti-Rabbit Ig Beads or TrueBlot™ anti-Goat Ig Beads ((Rockland, Limerick, PA). After several washes, beads were boiled and subjected to western blot analysis.

Construct generation: Plasmid containing full-length FAP and BIRC5 cDNA were obtained from Arizona State University (Cat #HsCD00040283 and Cat #HsCD00003606). FAP or BIRC5 cDNA was then PCR-amplified and subcloned into pCDH-CMV-MCS-EF1α-Puro (System Biosciences, Palo Alto, CA). To generate FAP/S624A mutant construct, point mutagenesis was performed using Q5® Site-Directed Mutagenesis Kit (New England Biolabs, Ipswich, MA). Primers used are as follows. All newly generated plasmids were subjected DNA sequence analysis to ensure the accuracy.

TABLE 2

| | FAP/S624A Point Mutagenesis Primer |
|---|---|
| | Nucleotide Sequence |
| SEQ ID NO: 29 | Forward: 5′-ATGGGGCTGGGCCTATGGAGG-3′ |
| SEQ ID NO: 30 | Reverse: 5′-ATGGCTATTCTTTTTTCATCAATG |
| | AAACCCATTTC-3′ |

Western blotting: Cells were harvested using RIPA Lysis and Extraction Buffer (Thermo Fisher) supplemented with Halt™ Proteinase and Phosphatase Inhibitor Cocktail (Thermo Fisher). Equal amounts of protein were loaded per lane into an SDS-PAGE gel followed by transferring onto a nitrocellulose membrane (BioRad). The blots were blocked with 5% nonfat dried milk followed by incubation in the respective antibodies overnight. After three washes, membranes were incubated with appropriate secondary antibodies and imaged. using Amersham Imager 600 Series (GE Healthcare. Chicago, IL). Antibodies used for western blot analysis include FAP (R&D systems, Cat #AF3715), Apoptosis Antibody Sampler Kit (CST, Cat #9915), p-AKT (CST, Cat #4060). AKT (CST. Cat #4685). NF-κB Family Antibody Sampler Kit II (CST. Cat #55764). GAPDH (CST, Cat #5174), PRKDC (Abcam, Cat #ab32566), PRMT5 (Abcam, Cat #ab109451).

Cell growth, in vitro invasion, flow Cytometry-based apoptosis and lipid raft isolation: Cell growth was analyzed using CCK-8 reagents (Sigma-Aldrich, Cat #96992). Briefly, cells were cultured in 96-well plates and CCK-8 reagent was added to cells. After 2-h incubation at 37° C., absorbance was measured using a Bio-Rad plate reader at 460 nm. In vitro invasion was assayed using Matrigel invasion chamber available from Cell Biolabs Inc (San Diego, CA) according to manufacturer's protocol. Flow cytometry-based cell apoptosis was determined using Dead Cell Apoptosis Kits with Annexin V (Thermo Fisher, Cat #V13242) according to manufacturer's protocol. Lipid raft fractions were prepared using Minute™ Plasma Membrane-Derived Lipid Raft Isolation Kit (Invent Biotechnologies, Plymouth, MN) according to manufacturer's protocol.

NF-κB promoter activity assay, NF-κB family member activation assay: NF-κB transcriptional activity was analyzed using NF-κB reporter gene construct from Promega (Madison, WI). To determine NF-κB activity, this plasmid was co-transfected into cells with a Renilla luciferase gene-containing plasmid (pRL-TK, Promega) for 36 h. Cells were lysed and cell lysates were assayed for luciferase activity using Dual-Luciferase® Reporter Assay System (Promega Corp. Madison. WI). Renella luciferase activity was used as an internal transfection control for standardization. NF-κB family member nuclear activation activity was analyzed using TransAM® NFκB Family kit (Active Motif, Carlsbad, CA) according to manufacturer's protocol.

Immunofluorescence staining: Cells were plated on coverslips for overnight and then fixed with 4% paraformaldehyde followed by permeabilization with 1% Triton X-100. Cells were washed three times with 1×PBS (Thermo Fisher, Cat #: 10010049). After brief blocking with 1% BSA, cells were incubated with anti-FAP mAb (Cat #sc-65398, Santa Cruz Biotechnology, Santa Cruz, CA) or anti-PRKDC polyclonal antibody (Cat #ab32566, Abcam) for 1 h and then with secondary antibody (Alexa Fluor® 488 and Alexa Fluor® 568) for another hour. Coverslips were mounted on coverslips using Permount solution (Cat #: P36961, Thermo Fisher) and cell fluorescence staining was viewed with confocal microscopy (Carl Zeiss Vision, LSM510). To locate lipid rafts, recombinant Cholera toxin subunit B conjugated with Alexa Fluor™ 647 (Cat #C34778, Thermo Fisher) was included during incubation with secondary antibody.

Generation of EpCAM-scramble and EpCAM-siFAP aptamer: The T7 promoter sequence was annealed with oligonucleotides containing either sense or antisense EpCAM aptamer-FAP siRNA sequence (or scrambled sequence) and then subjected to in vitro transcription using RiboMAX™ Large Scale RNA Production Systems (Promega). 2′-fluoro (F)-deoxycytidine and deoxyguanosine (TriLink Biotechnologies, San Diego, CA) were included in in vitro transcription reaction. After reactions, sense and antisense strands were annealed to generate aptamers. The configuration of aptamers s was analyzed using Forna package (http://rna.tbi.univie.ac.at/forna/). Oligonucleotides used for in vitro transcription are as follows.

TABLE 3

| | In vitro transcription |
|---|---|
| | nucleotide sequence |
| SEQ ID NO: 31 | T7 Promoter: 5′-AATTTAATA |
| | CGACTCACTATAG-3′ |
| SEQ ID NO: 32 | EpCAM Aptamer-Scrambled |
| | sense strand template: |
| | 5′-GGATGAACGAGGTATACGACT- |
| | TTACGACCGGGTAACCAGTCGCC- |
| | TATAGTGAGTCGTATTAAATT-3′. |
| SEQ ID NO: 33 | EpCAM Aptamer-Scrambled |
| | antisense strand |
| | template: |
| | 5′-CTTCATCACGTATAGTCGTCC- |
| | TTACGACCGGGTAACCAGTCGCC- |
| | TATAGTGAGTCGTATTAAATT-3′. |
| SEQ ID NO: 34 | EpCAM Aptamer-FAP siRNA |
| | sense strand template: |
| | 5′-CTTTGAAGGGCGTAAGACAATG |
| | C-TTACGACCGGGTAACCAGTCGCC- |
| | TATAGTGAGTCGTATTAAATT-3′ |
| SEQ ID NO: 35 | EpCAM Aptamer-FAP siRNA |
| | antisense strand |
| | template: |
| | 5′-GCATTGTCTTACGCCCTTCAA- |
| | TTACGACCGGGTAACCAGTCGCC- |
| | TATAGTGAGTCGTATTAAATT-3′ |

Mouse xenograft models, imaging, and drug administration: HEY and OVCAR8 cells (mesenchymal-like ovarian cancer cell lines) containing luciferase gene were intraperitoneally injected into 5-6-week-old nude female mice (1×106 cells/mouse) (Jackson Lab, Bar Harbor, ME) and intraperitoneal xenograft development was monitored weekly by measuring fluorescence in Xenogen IVIS-200 In Vivo Bioluminescence imaging system (PerkinElmer Inc, Waltham, MA). After 1 week of tumor cell injection, mice were randomized into two groups (5 per group) and intraperitoneally injected with EpCAM aptamer-scramble or EpCAM aptamer-FAP siRNA once every other days (5 nmole/mouse). At week 5, mice were sacrificed, and visible implants were collected from peritoneal cavities. Collected implants were weighed and aliquot of implants were also sent to UF Pathology core for immunohistochemistry staining of FAP, TUNEL and cleaved caspase-3. Animal studies and these procedures were approved by the Institution Animal Care Committee at University of Florida.

Data analysis: Patient data was retrieved from TCGA ovarian serous cystadenocarcinoma dataset containing 579 patients (TCGA_OV_gistic2thd2015-02-24). Volcano plot was generated using R software ggplot package (https://biocoreerg.github.io/CRG_RIntroduction/volcano-plots.html). Kaplan-Meier curves were generated using http://kmplot.com. All experiments were performed in triplicates. The results of each experiment are reported as the mean of experimental replicates. All statistical analyses were performed using PRISM 8.3 with unpaired t test (two-tailed). Error bars represent the standard deviation. For all tests, P<0.05 was considered significant.

### Example 2. FAP is Upregulated in Advanced Stages of Ovarian Cancer and Invasive Ovarian Cancer Cells

To identify candidate genes critical for ovarian cancer progression, we generated a volcano plot using TCGA ovarian cancer dataset to identify mRNA differentially expressed between patients in early (Stage I and II) and advanced stages (Stage III and IV) (Log 2 fold change >1.5 and P<0.05). Total of 24 genes were upregulated in advanced stages of patients while only one was upregulated (FIG. 1A and Table 5). We paid specific attention on FAP because it is a proteinase and regarded as targetable molecule. To explore FAP's relevance to ovarian cancer progression, we compared its expression between normal ovary tissue and ovarian cancer and found that FAP was much higher in ovarian cancer (FIG. 1B). Further analysis also showed that FAP expression is higher in Stage III and IV patients than Stage II patients (FIG. 1C). Kaplan-Meier analyses further showed that FAP was inversely correlated with patient survival (P=5.1×10-8 for overall survival and P=2.1×10-8 for progression free survival) (FIGS. 1D and 1E).

With the aid of established ovarian cancer cell lines, we previously show that mesenchymal-like, but not epithelial-like ovarian cancer cells are capable of undergo robust intraperitoneal xenograft development (Chen et al., 2010; Yang et al., 2015; Fang et al., 2017). Using E-cadherin as epithelial marker but vimentin as mesenchymal marker, we observed that all mesenchymal-like lines were highly invasive while epithelial-like lines barely invaded Matrigel (FIGS. 1F and 1G). Western blot analysis showed that FAP was only detected in invasive ovarian cancer cell lines (FIG. 1F), indicating that FAP is specifically involved in tumorigenic events in mesenchymal-like ovarian cancer.

### Example 3. FAP is Involved in Ovarian Cancer Cell Survival Independent of its Proteinase Function

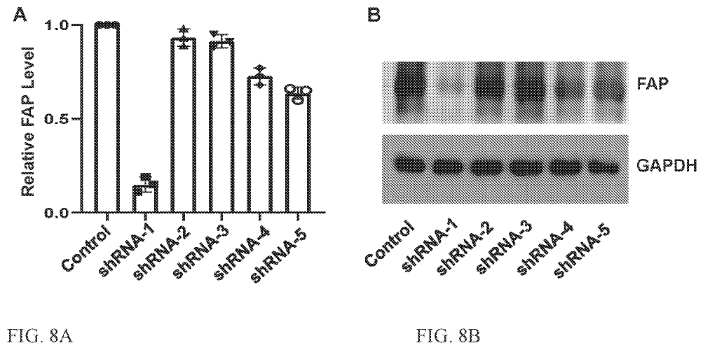

To investigate how silencing FAP impact on ovarian cancer behaviors, we screened a set of FAP shRNAs in HEY cells and were able to identify only one effectively depleting FAP (FIG. 8). Lentivirally transducing this shRNA led to massive cell death in invasive ovarian cancer cell lines (FAP-expressing) while survival of non-invasive lines (FAP-negative) was not affected (FIG. 2A). To rule out the off-target effect of shRNA and determine the importance of FAP's proteinase function for cell survival, we introduced either wild-type or proteinase mutant of FAP into FAP-knockdown cells. Cell viability assay (CCK8) showed that both similarly abrogated cell death trigged by FAP shRNA (FIG. 2A). These results not only confirmed the specificity of FAP knockdown effect but also indicate that proteinase activity is dispensable for FAP regulation of cell survival. This notion is further supported by the observation that metalloprotease inhibitor Batimastat and dipeptidase inhibitor Alabostat either alone or together displayed little effect on survival of HEY and OVCAR8 cells (FIG. 9).

To determine whether FAP-knockdown triggered apoptosis, we performed Annexin V/propidium iodine (PI) staining-based flow cytometry. In control HET and OVCAR8 cells, approximately 12.7 and 11.67% of total cell population were apoptotic (Q2+Q3) (FIG. 2C). In contrast, these percentages rose to 57.4 and 80.4% respectively in FAP-knockdown cells (FIG. 2C). Parallel western blot showed much more cleaved PARP and cleaved caspase-3 appeared in FAP-knockdown cells than those in control (FIG. 2D). Moreover, we found that both pan-caspase inhibitor zVAD and caspase-3 inhibitor DEVD blocked FAP shRNA-led cell death (FIG. 2E). Collectively, these results suggest that silencing FAP results in apoptosis of invasive ovarian cancer cells.

### Example 4. The Presence of FAP is Essential for Robust NF-κB Activity in Ovarian Cancer Cells

To elucidate molecular mechanism underlying FAP regulation of cell survival, we performed RNAseq from both control and FAP-knockdown OVCAR8 cells followed by Gene Sequence Enrichment Analysis (GSEA) and Ingenuity Pathway Analysis (IPA). Both analyses pinpointed that tumor necrosis factor receptor (TNFR) signaling was impaired in FAP-knockdown cells (FIGS. 3A and 3B). As TNFR signaling is well established to promote cell survival by promoting NF-κB activity (5), we measured NF-κB activity by introducing luciferase reporter gene plasmid containing 5×NFκB binding sequence into both control and FAP-knockdown cells. By judging luciferase activity, we detected over 80% reduction in NF-κB activity in FAP-knockdown HEY and OVCAR8 cells in comparison with their respective controls (FIG. 3C). Further ELISA-based NF-κB transactivation assay showed marked reduction of REL binding to oligonucleotide containing NF-κB consensus sequence in FAP-knockdown HEY and OVCAR8 cells (FIG. 3D and FIG. 10A).

To define the cause of less REL binding in FAP-knockdown cells, we detected dramatic reduction of both REL mRNA and protein in FAP-knockdown cells (FIGS. 3E and 3F). In contrast, knockdown of FAP did not alter the subcellular localization of REL (FIG. 10B). To confirm that reduced NF-κB activity in FAP-knockdown cells was relevant to REL, we showed that silencing REL exhibited similar inhibitory effect in NF-κB activity as FAP knockdown was able to do (FIG. 3G). In addition, we showed that only silencing REL (but not RELA or RELB) was able to decrease cell viability (FIG. 3H). These results suggest that FAP is essential for survival of invasive ovarian cancer cells by sustaining REL-associated NF-κB activity.

### Example 5. FAP-REL Axis is Involved in the Survival of Ovarian Cancer Cells by Sustaining BIRC5 (Survivin) Abundance

NF-κB signaling promotes cell survival by maintaining the expression of various pro-survival proteins (6). We thus examined the levels of known NF-κB-regulated pro-survival proteins between control and FAP-knockdown cells. Western blot analysis showed that knockdown of FAP diminished BIRC5 but did little on others (FIG. 4A). Similarly, knockdown of REL also abrogated BIRC5 in both HEY and OVCAR8 cells (FIG. 4B).

To functionally link the disappearance of BIRC5 to cell death elicited by FAP or REL knockdown, we introduced BIRC5 shRNAs into HEY and OVCAR8 cells followed by analyzing cell viability. CCK8 assay showed that knockdown of BIRC5 evidently reduced cell number (FIG. 4C). Similarly, treatment of BIRC5 inhibitor YM155 led to significant decrease in cell viability while BIRC2 inhibitor Birinapant and Mcl1 inhibitor S63845 displayed much less inhibitory effect (FIG. 4D).

When BIRC5 was ectopically expressed in HEY or OVACR8 cells, we found that FAP or REL shRNA lost their ability to suppress cell viability (FIG. 4E). These results suggest that FAP-REL signaling axis promotes ovarian cancer cell survival by sustaining BIRC5 expression.

### Example 6. PRKDC is Involved in Ovarian Cancer Cell Survival

To uncover the link between FAP and NF-κB activation, we generated FAP immunoprecipitate from OVCAR8 cells followed by mass spectrometry analysis to identify FAP interacting proteins. As expected. FAP had the most hits from the immunoprecipitate (Table 6). Among other proteins with reasonable hits, we focused on PRMT5 and PRKDC (Table 6) as both have been reported to regulate NF-κB activity (7, 8). We introduced PRMT5 or PRKDC shRNAs into HEY and OVCAR8 cells and found that knockdown of PRKDC, but not PRMT5 reduced cell viability (FIGS. 5A and S5A) and NF-κB activity (FIGS. 5B and S5B). Western blot analysis further showed that knockdown of PRKDC abolished REL and BIRC5 but exhibited much less effect on FAP (FIG. 5C). In contrast, knockdown of PRMT5 did little on the abundance of FAP, REL or BIRC5 in both HEY and OVCAR8 cells (FIG. S5C).

PRKDC has been shown to promote cell survival involving AKT phosphorylation (Lu et al., 2006; Bozulic et al, 2008) while Akt signaling pathway is well characterized for its importance in NF-κB activation (Ozes et al, 1999; Madrid et al, 2001). We analyzed Akt phosphorylation in control and PRKRC-knockdown HEY and OVCAR8 cells and observed that level of Akt phosphorylation was almost completely abolished in PRKDC-knockdown cells (FIG. 5D). Similarly, silencing FAP also greatly lowered the abundance of phosphorylated Akt (FIG. 5E). As Akt inhibitor diminished NF-κB activity in both HEY and OVCAR8 cells (FIG. 5F), these results support the notion that PRKDC acts as a function connection between FAP and NF-κB activation in ovarian cancer cells.

### Example 7. FAP Directly Interacts with PRKDC and Key for PRKDC Localization in Lipid Rafts

To provide mechanistic basis of PRKDC in FAP regulation of NFκB activation in ovarian cancer cells, we initially performed co-immunoprecipitation to determine if FAP and PRKDC interacted using antibody against FAP or PRKDC. FAP was detected in PRKDC immunoprecipitates while PRKDC was seen in FAP immunoprecipitates (FIG. 6A). Subsequent confocal microscope-based immunofluorescence staining further revealed the co-localization of FAP and PRKDC in both HEY and OVCAR8 cells and the interaction appeared to be only present in the particular areas of inner plasma membrane (FIG. 6B).

Both FAP and PRKDC have been reported to be present in the lipid rafts with unknown function (13, 14). We investigated whether FAP-PRKDC interaction occurred in lipid rafts. We first isolated lipid fraction from HEY and OVCAR8 cells followed by western blot analysis to detect FAP and PRKDC. As well characterized lipid raft marker caveolin-1, both FAP and PRKDC were readily seen in lipid raft isolates (FIG. 6C). Subsequently, we performed con-focal immunofluorescence staining including lipid raft marker cholera toxin B (CT-B) and spotted that FAP-PRKDC interaction was co-localized with CT-B staining (FIG. 6D). However, disruption of lipid rafts using MBC yielded undetectable FAP-PRKDC co-localization in both cell lines (FIG. 6E). These data indicate that FAP-PRKDC interaction only occurs in lipid rafts.

Lipid raft has long been recognized as a site to elicit signaling. We hypothesized that FAP recruits PRKDC to lipid raft to launch its signaling for NF-κB activation. To test this possibility, we analyzed the amount of PRKDC in lipid raft fraction of HEY and OVCAR8 cells. Western blot revealed little or no PRKDC in FAP-knockdown cells while the level of caveolin is unaltered between control and FAP-knockdown cells, suggesting that absence of FAP prevents PRKDC recruitment to lipid raft but not formation of lipid rafts (FIG. 6F). Further confocal immunofluorescence staining showed that robust PRKDC staining with cholera toxin staining which such co-staining was not detected in FAP-knockdown cells (FIG. 6G). These results support the notion that FAP recruits PRKDC to lipid rafts, leading to NF-κB activation in ovarian cancer cells.

### Example 8. EpCAM Aptamer-Delivered FAP Aptamer Effectively Suppresses Intraperitoneal Ovarian Cancer Development

EpCAM is highly expressed in ovarian cancer but not in other tissues in peritoneal cavity, and an EpCAM aptamer was selected as the vehicle for FAP siRNA delivery. In this end, an EpCAM aptamer-FAP siRNA chimera was designed in which both ends are EpCAM aptamers and middle portion is FAP siRNA or scrambled sequence (FIG. 7A). When HEY and OVCAR8 cells were treated with control (scrambled sequence) or FAP siRNA-containing aptamer, it was found that aptamer-delivered FAP siRNA reduced approximately 80% of cell viability compared with the control (FIG. 7B).

TABLE 4

| | RNA nucleotide sequence |
|---|---|
| SEQ ID NO: 1 | EpCAM aptamer: |
| | 5′-GGCGACUGGUUACCCGGUCGUAA-3′ |
| SEQ ID NO: 2 | Reverse nucleotide sequence |
| | of EpCAM aptamer: |
| | 5′-AAUGCUGGCCCAUUGGUCAGCGG-3′ |
| SEQ ID NO: 3 | Anti-FAP siRNA sense: |
| | 5′-GCAUUGUCUUACGCCCUUCAAAG-3′ |
| SEQ ID NO: 4 | Anti-FAP siRNA antisense: |
| | 5′-UUGAAGGGCGUAAGACAAUGC-3′ |

To evaluate the efficacy of EpCAM aptamer-delivered FAP siRNA to impede intraperitoneal xenograft development of ovarian cancer cells, female athymic nude mice were intraperitoneally injected with luciferase-expressing HEY or OVCAR8 cells. After 1 week of tumor cell injection, scramble control or FAP siRNA-containing aptamer was administered at 5 nmole/mouse once every other day. Weekly bioluminescence imaging showed that both HEY and OVCAR8 cells underwent effective intraperitoneal xenograft development in mice receiving control aptamer (FIGS. 7C and 7D). In contrast, administrating FAP siRNA-containing aptamer deterred this process (FIG. 7C and FIG. 7D). At 5 weeks of post-treatment, all mice were euthanized and peritoneal metastatic colonization was visualized. Tumor implants were much less and smaller in mice receiving FAP siRNA-containing aptamer than those in mice administered with control aptamer (FIG. 7E). To quantitate the difference, tumor implants were harvested from sacrificed mice. Tumor implant weights were significantly less in FAP siRNA-treated mice than in control-treated ones (FIG. 7F).

To link impediment of peritoneal xenograft development to diminished FAP expression and apoptosis, IHC was performed to examine the intensity of FAP, cleaved CASP3 and TUNEL staining on harvested tumors. Strong FAP but no cleaved CASP3 or TUNEL were detected in tumors excised from control aptamer-treated mice (FIG. 7G). In contrast, tumors derived from FAP siRNA-containing aptamer-treated mice displayed little FAP but robust cleaved CASP3 and TUNEL staining (FIG. 7G). These results provide pre-clinic evidence that EpCAM aptamer-delivered FAP siRNA can potentially be an effective therapeutic tool against mesenchymal-like (invasive) ovarian cancer.

As will be apparent to one of ordinary skill in the art from a reading of this disclosure, further embodiments of the present invention can be presented in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. Such equivalents are considered to be within the scope of this invention. Numerous changes in the details of implementation of the invention can be made without departing from the spirit and scope of the invention, which is limited only by the claims that follow. Features of the disclosed embodiments can be combined and rearranged in various ways within the scope and spirit of the invention. The scope of the invention is as set forth in the appended claims and equivalents thereof, rather than being limited to the examples contained in the foregoing description.

### REFERENCES

All references listed below and throughout the specification are hereby incorporated by reference in their entirety.
1. Aggarwal S, Brennen W N, Kole T P, et al., Fibroblast activation protein peptide substrates identified from human collagen I derived gelatin cleavage sites. Biochemistry (2008) 47:1076-86.
2. Fitzgerald A A and Weiner L M., The role of fibroblast activation protein in health and malignancy, Cancer Metastasis Rev. (2020) 39(3):783-803.
3. Baeuerle P A and Gires O, EpCAM (CD326) finding its role in cancer. Br J Cancer (2007) 96:417-423.
4. Balzar M, Winter M J, de Boer C J, and Litvinov S V, The biology of the 17-1A antigen (Ep-CAM). J Mol Med (1999) 77:699-712.
5. Basu S, Rosenzweig K R, Youmell M, and Price B D, The DNA-dependent protein kinase participates in the activation of NF kappa B following DNA damage. Biochemical and biophysical research communications (1998) 247(1):79-83.
6. Bozulic L, Surucu B, Hynx D, and Hemmings B A, PKB alpha/Akt1 acts downstream of DNA-PK in the DNA double-strand break response and promotes survival. Mol Cell. (2008) 30(2):203-13.
7. Brenner D, Blaser H, and Mak T W, Regulation of tumour necrosis factor signalling: live or let die. Nat Rev Immunol. (2015) 15(6):362-74.
8. Chen H, Wu X, Pan Z K, and Huang S, Integrity of SOS1/EPS8/ABI1 tri-complex determines ovarian cancer metastasis. Cancer research (2010) 70(23):9979-90.
9. Chen Y and Huang L, Tumor-targeted delivery of siRNA by non-viral vector: safe and effective cancer therapy. Expert Opin Drug Deliv. (2008) December; 5(12):1301-1311.
10. Cirulli V. Crisa L, Beattie G M, et al., KSA antigen Ep-CAM mediates cell-cell adhesion of pancreatic epithelial cells: morphoregulatory roles in pancreatic islet development. J Cell Biol. (1998) 140:1519-1534.
11. Collins P J. McMahon G, O'Brien P, and O'Connor B, Purification, identification and characterisation of seprase from bovine serum. Int J Biochem Cell Biol (2004) 36:2320-2333.
12. Edosada C Y. Quan C. Tran T, et al., Peptide substrate profiling defines fibroblast activation protein as an endopeptidase of strict Gly 2-Pro 1-cleaving specificity. FEBS Lett (2006) 580:1581-1586.
13. Fang D, Chen H, Zhu J Y, et al., Epithelial-mesenchymal transition of ovarian cancer cells is sustained by Rac1 through simultaneous activation of MEK1/2 and Src signaling pathways. Oncogene (2017) 36(11):1546-58.
14. Goldstein L A, Ghersi G, Pineiro-Sanchez M L, et al., Molecular cloning of seprase: a serine integral membrane protease from human melanoma. Biochim Biophys Acta (1997) 1361:11-19
15. Huang C-H, Suen C-S, Lin C-T, et al., Cleavage-site specificity of prolyl endopeptidase FAP investigated with a full-length protein substrate. J Biochem (2011) 149:685-692.
16. Huang Y. Simms A E, Mazur A, et al., Fibroblast activation protein-α promotes tumor growth and invasion of breast cancer cells through non-enzymatic functions. Clin Exp Metastasis (2011) 28:567-579.
17. Knopf J D, Tholen S, Koczorowska M M, et al., The stromal cell-surface protease fibroblast activation protein-alpha localizes to lipid rafts and is recruited to invadopodia. Biochim Biophys Acta. (2015) 1853(10 Pt A):2515-25.
18. LaCasse E C, Baird S, Korneluk R G, and Mackenzie A E, The inhibitors of apoptosis (IAPs) and their emerging role in cancer. Oncogene (1998) 17(25):3247-59.
19. LARGE MOLECULE THERAPEUTICS|Oct. 5, 2015, Gene Knockdown by EpCAM Aptamer-siRNA Chimeras Suppresses Epithelial Breast Cancers and Their Tumor-Initiating Cells
20. Lee K N, Jackson K W, Christiansen V J, et al., Antiplasmin-cleaving enzyme is a soluble form of fibroblast activation protein. Blood (2006) 107:1397-404.
21. Litvinov S V, Balzar M, Winter M J, et al., Epithelial cell adhesion molecule (Ep-CAM) modulates cell-cell interactions mediated by classic cadherins. J Cell Biol. (1997) 139:1337-1348.
22. Lu D, Huang J and Basu A, Protein kinase Cepsilon activates protein kinase B/Akt via DNA-PK to protect against tumor necrosis factor-alpha-induced cell death. Journal of Biological Chemistry 2006; 281(32):22799-807.
23. Lucero H, Gae D, and Taccioli G E, Novel localization of the DNA-PK complex in lipid rafts: a putative role in the signal transduction pathway of the ionizing radiation response. Journal of Biological Chemistry (2003) 278(24):22136-43.
24. Lv B, Xie F. Zhao P, et al., Promotion of Cellular Growth and Motility Is Independent of Enzymatic Activity of Fibroblast Activation Protein-α. Cancer Genomics Proteomics (2016) 13:201-8
25. Madrid L V, Mayo M W, Reuther J Y, and Baldwin A S, Jr., Akt stimulates the transactivation potential of the RelA/p65 Subunit of NF-kappa B through utilization of the Ikappa B kinase and activation of the mitogen-activated protein kinase p38. Journal of Biological Chemistry (2001) 276(22):18934-40.
26. Okegawa T, Pong R C, Li Y, and Hsieh J T, The role of cell adhesion molecule in cancer progression and its application in cancer therapy. Acta Biochim Pol. (2004) 51(2):445-57
27. Osta W A, Chen Y, Mikhitarian K, et al., EpCAM is overexpressed in breast cancer and is a potential target for breast cancer gene therapy. Cancer Res. (2004) 64:5818-5824.
28. Ozes O N, Mayo L D, Gustin J A, et al., NF-kappaB activation by tumour necrosis factor requires the Akt serine-threonine kinase. Nature (1999) 401(6748):82-5.
29. Ramirez-Montagut T, Blachere N E, Sviderskaya E V, et al., FAPα, a surface peptidase expressed during wound healing, is a tumor suppressor. Oncogene (2004) 23:5435-5446.
30. Rosenblum J S and Kozarich J W, Prolyl peptidases: a serine protease subfamily with high potential for drug discovery. Curr Opin Chem Biol (2003) 7:496-504.
31. Society A C. Cancer Facts & Figures 2019. Atlanta, GA: American Cancer Society; 2019.
32. Sun S, Albright C F, Fish B H, et al., Expression, Purification, and Kinetic Characterization of Full-Length Human Fibroblast Activation Protein. Protein Expr Purif (2002) 24:274-281.
33. Trzpis M, Mclaughlin P M J, de Leij L M F H, and Harmsen M C, Epithelial Cell Adhesion Molecule More than a Carcinoma Marker and Adhesion Molecule, Am J Pathol. (2007) August; 171(2):386-395.
34. Nochi T, Yuki Y, Terahara K, et al., Biological role of Ep-CAM in the physical interaction between epithelial cells and lymphocytes in intestinal epithelium. Clin Immunol. (2004) 113:326-339.
35. Wei H, Wang B, Miyagi M, et al., PRMT5 dimethylates R30 of the p65 subunit to activate NF-kappaB. Proceedings of the National Academy of Sciences of the United States of America. (2013) 110(33):13516-21.
36. Xiang D, Zheng C, Zhou S, and Qiao S, Superior Performance of Aptamer in Tumor Penetration over Antibody: Implication of Aptamer-Based Theranostics in Solid Tumors, Theranostics (2015) 5(10):1083-1097.
37. Yang L, Fang D, Chen H, et al., Cyclin-dependent kinase 2 is an ideal target for ovary tumors with elevated cyclin E1 expression. Oncotarget. (2015); 6(25):20801-12.

What is claimed is:

1. A FAP silencing oligonucleotide comprising an anti-FAP siRNA comprising
(1) a sense oligonucleotide of SEQ ID NO:3 or a biologically active fragment or variant thereof, and
(2) an antisense oligonucleotide of SEQ ID NO:4 or a biologically active fragment or variant thereof,
wherein the sense oligonucleotide and antisense oligonucleotide comprise 2′-fluoro-deoxyguanosine or natural deoxyguanosine.

2. The FAP silencing oligonucleotide comprising an anti-FAP siRNA according to claim 1, wherein the anti-FAP siRNA is linked at both ends to an aptamer that recognize cancer markers on the cancer cell surface, wherein the aptamer linked at both ends can be the same aptamer or different aptamer.

3. The FAP silencing oligonucleotide comprising an anti-FAP siRNA according to claim 1 or 2, wherein the aptamer is EpCAM recognizing aptamer.

4. The FAP silencing oligonucleotide comprising an anti-FAP siRNA according to claim 3, wherein the EpCAM recognizing aptamer is SEQ ID NO:1 or SEQ ID NO:2.

5. The FAP silencing oligonucleotide comprising an anti-FAP siRNA according to claim 4, wherein
(1) the sense oligonucleotide is SEQ ID NO:5 or a biologically active fragment or variant thereof, and
(2) the antisense oligonucleotide is SEQ ID NO:6 or a biologically active fragment or variant thereof.

6. The FAP silencing oligonucleotide comprising an anti-FAP siRNA according to claim 4, wherein
(1) the sense oligonucleotide is SEQ ID NO:7 or a biologically active fragment or variant thereof, and
(2) the antisense oligonucleotide is SEQ ID NO:8 or a biologically active fragment or variant thereof.

7. The FAP silencing oligonucleotide comprising an anti-FAP siRNA according to claim 4, wherein
(1) the sense oligonucleotide is SEQ ID NO:9 or a biologically active fragment or variant thereof, and
(2) the antisense oligonucleotide of SEQ ID NO:10 or a biologically active fragment or variant thereof.

9. The FAP silencing oligonucleotide comprising an anti-FAP siRNA according to claim 4, wherein
(1) the sense oligonucleotide is SEQ ID NO:11 or a biologically active fragment or variant thereof, and
(2) the antisense oligonucleotide is SEQ ID NO:12 or a biologically active fragment or variant thereof.

10. A pharmaceutical composition for the treatment of cancer susceptible to FAP silencing, comprising a pharmaceutically acceptable carrier and at least one of FAP silencing oligonucleotides according to any one of claims 1, 6-9 or combination thereof.

11. A method for treating a cancer susceptible to FAP silencing in a subject in need, comprising administering a therapeutically effective amount of FAP silencing oligonucleotides of any one of claims 1, 6-9 or combination thereof.

12. The method of claim 11, wherein the cancer is colon adenocarcinoma, esophagus adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, breast cancer, ductal carcinoma, lobular carcinoma, melanoma, intraductal carcinoma, mucinous carcinoma, phyllodes tumor, ovarian cancer including ovarian adenocarcinoma, endometrium adenocarcinoma, granulose cell tumor, mucinous cystadenocarcinoma, cervix adenocarcinoma, vulva squamous cell carcinoma, basal cell carcinoma, prostate cancer, giant cell tumor of bone, bone osteosarcoma, larynx carcinoma, lung adenocarcinoma, kidney carcinoma, urinary bladder carcinoma, Wilm's tumor, uterine cancer, endometrial cancer and lymphoma.

13. The method of claim 12, wherein the cancer is ovarian cancer.

14. The method of claim 11, further comprising co-administering a therapeutically effective amount of an adjunct cancer therapeutic agent,
wherein the adjunct cancer therapeutic agent comprises an antitumor alkylating agent, antitumor antimetabolite, antitumor antibiotics, plant-derived antitumor agent, antitumor platinum complex, antitumor camptothecin derivative, antitumor tyrosine kinase inhibitor, monoclonal or polyclonal antibody, interferon, biological response modifier, hormonal anti-tumor agent, anti-tumor viral agent, angiogenesis inhibitor, differentiating agent, PI3K/mTOR/AKT inhibitor, cell cycle inhibitor, apoptosis inhibitor, hsp 90 inhibitor, tubulin inhibitor, DNA repair inhibitor, antiangiogenic agent, receptor tyrosine kinase inhibitor, topoisomerase inhibitor, taxane, agent targeting Her2, hormone antagonist, agent targeting a growth factor receptor, or a pharmaceutically acceptable salt thereof.

15. The method of claim 11, further comprising treating FAP silencing-susceptible cancer with at least one adjunct cancer therapy protocol selected from the group consisting of surgery, radiation therapy, chemotherapy, gene therapy, DNA therapy, adjuvant therapy, neoadjuvant therapy, viral therapy, RNA therapy, immunotherapy, and nanotherapy.
